(19) United States
(12) Patent Application Publication
(10) Pub. No.: US 2008/0279879 A1
(43) Pub. Date: Nov. 13, 2008

(54) INDUCTION OF BROADLY REACTIVE NEUTRALIZING ANTIBODIES BY FOCUSING THE IMMUNE RESPONSE ON V3 EPITOPES OF THE HIV-1 gp120 ENVELOPE

(75) Inventor: Susan Zolla-Pazner, New York, NY (US)

Correspondence Address: BROWDY AND NEIMARK, P.L.L.C.;624 NINTH STREET, NW, SUITE 300, WASHINGTON, DC, 20001-5303, US

(73) Assignee: New York University, New York, NY (US)

(21) Appl. No.: 11/942,634

(22) Filed: Nov. 19, 2007

Related U.S. Application Data
(60) Provisional application No. 60/859,486, filed on Nov. 17, 2006.

(51) Int. Cl. A61K 39/21; A61P 31/18

(52) U.S. Cl. 424/188.1; 424/192.1

(57) ABSTRACT

Compositions, kits and methods for boosting, or for priming and boosting, high titer broadly neutralizing cross-clade antibody responses focused on single HIV-1 neutralizing epitopes are disclosed. gp120 DNA plasmids comprising HIV env genes are used to prime the antibody response. Primed subjects are immunized with recombinant fusion proteins that comprise a “carrier” protein fusion partner, preferably a truncated form of the MuLV gp70 Env protein, and a desired HIV neutralizing epitopes. Preferred epitopes are epitopes of V3 from one or more HIV clades. Immune sera from such immunized subjects neutralized primary isolates from virus strains heterologous to those from which the immunogens were constructed. Neutralizing activity was primarily due to V3-specific antibodies and cross-clade neutralizing Abs were present. This approach results in more potent and broader neutralizing antibody levels, a result of “immunofocusing” the humoral immune response on neutralizing epitopes such as V3.

### STATEMENT OF RIGHTS TO INVENTIONS MADE UNDER FEDERALLY SPONSORED RESEARCH

This invention was funded in part by a grant from the National Institutes of Health (AI36085) which provides to the United States government certain rights in this invention.

### BACKGROUND OF THE INVENTION

1. Field of the Invention

The invention in the field of biochemistry and medicine relates to improved HIV envelope protein (Env) immunogen or vaccine compositions and methods that focus the neutralizing antibody (“Ab”) response on selected viral epitopes such as V3 neutralizing epitopes.

2. Description of the Background Art

Protective antibodies (Abs) are needed to reduce the size of a virus inoculum and block infection of target cells. The ability of Abs to afford such protection against HIV-1 (also referred to herein as “HIV”) has been documented by several passive immunization studies in animals and by recent results suggesting that neutralizing Abs (“NAbs”) in HIV-infected individuals protect against superinfection (Smith, D M et al., 2006, Virology 355:1-5)

Since sera from some HIV-infected individuals have broad neutralizing activity (Pilgrim, A K (1997) J Infect Dis. 176):924-32; Nyambi, P N et al. (1996) J Virol. 70:6235-43) and several anti-HIV monoclonal Abs (mAbs) neutralize a broad spectrum of primary isolates (Binley J M et al. (2004) J Virol. 78:13232-52), it is clear that the human B cell repertoire includes genes capable of encoding Abs that can recognize and neutralize a broad spectrum of HIV isolates. Epitopes that are known to induce broadly neutralizing Abs include the membrane proximal region of gp41, the CD4 binding site on gp120, complex glycans on gp120, CD4-induced epitopes in and around the gp120 bridging sheet, and the V3 loop of gp120 (reviewed in Gorny M K et al. (2004) J Virol. 78:2394-404).

Despite the extensive information on HIV NAbs, it has proven difficult to induce broadly neutralizing Ab responses against HIV by immunization. This is due to several factors including the poor immunogenicity of Env proteins (Wyatt, R et al. (1998) Science 280(5371): 1884-8; Flynn, N M (2005) J Infect Dis 191:654-65) the predominant induction of non-neutralizing rather than neutralizing Abs (Belshe, R B et al. (1993) J Infect Dis. 168:1387-95; Parren, P W et al. (1997) Nat Med 3:366), the masking of neutralization-sensitive epitopes (Fox D G et al. (1997) J Virol. 71:759-65; Kwong P D et al. (2002) Nature 420:678-82; Wei, X et al. (2003) Nature 422:307-23; Krachmarov C P et al. (2006) J Virol. 80:7127-35), the high mutation rate of HIV leading to antigenic variability, and to a myriad of other factors such as the physicochemical characteristics of the virus membrane (Harada, S et al. (2005) Biochem Biophys Res Commun 329: 480-6), and the affinity of the Env for CD4 (Hammond, A L et al. (2001) J Virol. 75:5593-603).

A variety of forms of HIV Env have been used as immunogens, including the gp120, gp140 and gp160 forms of the Env glycoprotein, various oligomeric constructs, and several complete and truncated forms for Env expressed as components of recombinant viruses). Nonetheless, the best, way to generate cross-clade NAb responses, albeit modest ones, have utilized strategies in which a DNA Env expression vector was given as a priming immunogen and either a recombinant adenovirus or an Env protein was used as a boosting immunogen. The promise of this approach was first demonstrated with constructs derived from T cell line-adapted HIV strains (Richmond J F et al. (1998) J Virol. 72:9092-9100; Lu, S et al., (1998) AIDS Res Hum Retroviruses 14:151-5) and has more recently been confirmed and extended using HIV env genes derived from various HIV strains including primary isolates (Richmond et al., supra); Wang, S et al., (2006). Virology 350:34-47; Barnett, S W et al. (2001) J Virol 75:5526-40; Barnett, S W et al. (1997) Vaccine 15:869-73; Beddows, S et al. (2005) J Virol 79:8812-27; Wang, S et al. (2005). J Virol 79:7933-7). Thus, in rabbits, priming with gp120_{JF-FL} DNA and boosting with Env_{JR-FL} induced NAbs to the relatively resistant homologous strain as well as to a limited number of other clade B primary isolates (Wang et al., 2005, supra). Priming with a polyvalent cocktail of gp120 DNA plasmids and boosting with a cocktail of Env proteins from various clades induced a broader response, with cross-clade NAbs to strains from clades A, C, D, and E (Wang et al., 2006, supra); Lian Y et al. (2005) J Virol. 79:13338-49). Similarly, in guinea pigs, a polyvalent env DNA prime, and a polyvalent boost with recombinant adenoviruses each carrying env genes from clades A, B and C, resulted in Abs able to neutralize strains from these clades, albeit at titers of only 1:5 (Chakrabarti B K et al. (2005) Vaccine 23:3434-45). In each of these experiments, the boosting immunogen was a form of gp120 or modified gp140 proteins, immunogens which contain a multitude of B cell epitopes.

An alternative immunization approach has been the construction and use of an immunogen that will focus the immune response on one or a few epitopes that are known to induce neutralizing Abs. An advantage of this approach is the potential to induce an immune response with a larger proportion, and consequently a higher titer of, neutralizing Abs. The use of selected epitopes or mimotopes for the construction of vaccines that preferentially induce protective Abs is still in its infancy, although some striking examples exist, especially with polysaccharide antigens of various pathogens (Beenhouwer, D O et al., (2002) J Immunol 169:6992-9; Buchwald, U K et al. (2005) Infect Immun 73:325-33). In the search for an HIV vaccine, several attempts have been made to graft a neutralizing epitope from the virus envelope into a foreign protein. For example, the neutralizing epitope in the membrane proximal external region of gp41 recognized by human mAb 2F5 has been grafted into influenza virus and the hepatitis B surface antigen (Eckhart, L et al. (1996) J Gen Virol 77:2001-8; Muster, T et al. (1995). J Virol 69:6678-86). However these constructs failed to induce neutralizing Abs. In contrast, a peptide mimotope selected on the basis of binding to the broadly neutralizing human anti-V3 mAb 447, when covalently conjugated to a protein carrier induced an Ab response which, although limited in potency and breadth, could neutralize two virus strains (Keller, P M et al. (1993). Virology 193:709-16). Another strategy, using a recombinant protein prime and boosters containing V2 and V3 peptides resulted in increased titers of anti-peptide Abs and an increase in serum neutralizing Abs for the homologous and related virus (Davis, D. et al. (1997) Vaccine 15:1661-9).

Conventional wisdom suggested to the present inventor that “constant” or conserved rather than “variable” (V) regions of HIV-1 Env glycoproteins should induce the most broadly reactive Abs. However, of the several epitopes in the conserved regions of gp120 and gp41 that induce NAbs, all are concealed or “protected” by protein folding, glycosylation, and/or oligomerization of the Env proteins on the virus surface. Most epitopes of Env proteins are only transiently exposed during the process of infection or are poorly immunogenic. In contrast, the V region (or V loop) known as V3 appears to be at least partially exposed during various stages of the infectious process, is immunogenic in essentially all HIV+ subjects, and is capable of inducing NAbs that can neutralize a broad array of primary isolates.

This cross-reactivity of V3 is counter-intuitive if one considers only the sequence variability rather than on conserved nature of V3 structures which must be present in order to mediate selection of, and interaction with, chemokine receptors. The present inventor has turned her attention to the structural conservation of the V3 loop instead of its sequence variability, which led logically to the concept that Abs to one V3 loop can cross-react with others. This is, in fact a logical outgrowth of the classic immunochemical studies of Landsteiner, Heidelberger and Kabat which showed that cross-reactive Abs recognize antigens which possess similar structural groupings. (See, for example, Kabat, E A and Mayer, M M, Experimental Immunochemistry. Charles C. Thomas, Springfield, Ill., 1961.)

Generating anti-HIV-1 neutralizing antibodies remains a major scientific challenge for HIV-1 vaccine development. Phase III trial of gp120 vaccine immunogens represent the only antibody-based vaccine candidate to be tested for efficacy in humans.

One hundred seventy-four human anti-Env human mAbs and Fab fragments are listed in the Los Alamos Immunology Database, and these have been useful for defining the human Ab response to Env and identifying which Abs possess neutralizing activity. Although these mAbs recognize 11 Env regions, only mAbs specific for 4 regions of gp120 and gp41 are capable of broad and potent virus neutralization. These include (a) one region in gp41, the membrane-proximal region (“MPR”), and (b) three regions in gp120: (i) the CD4 binding site (“CD4bs”), (ii) complex carbohydrate moieties on the outer face of gp120, and (iii) the chemokine receptor binding region which consists of portions of the V1V2 stem, V3, and C4 domains (Nabel, G. J. 2005. Science 308:1878). Each region presents problems for vaccine design, as explained below.
(1) The gp41 MPR is poorly immunogenic and has so far failed to induce NAbs when introduced into several constructs. Moreover, the two neutralizing mAbs that do target this region were shown to cross-react (autoreact) with “normal” human antigens (Haynes, B F et al., 2005. Science 308: 1906-8).
(2) Although the CD4bs is highly immunogenic, only one of many anti-CD4bs mAbs has neutralizing activity, suggesting that this is not an epitope that preferentially induces protective Abs. The single neutralizing anti-CD4bs mAb is also autoreactive (Haynes et al., supra).
(3) The aforementioned carbohydrate epitope on gp120 is poorly immunogenic and has been defined by only a single mAb, which has an aberrant structure that is probably extremely rare in the human Ab repertoire (Zwick, M B et al. (2003) J Virol 77:5863-78).
(4) Finally, the HIV chemokine receptor binding region is targeted by at least two distinct sets of mAbs: (a) those that recognize the bridging sheet—the so-called “CD4-induced” (“CD4i”) Abs—and (b) those that target V3. The former, though readily produced in infected individuals, are limited as prototypes for vaccine-induced Abs because, essentially, only their Fab fragments can neutralize primary HIV isolates (Labrijn, A F et al. (2003) J Virol 77:10557-65). The anti-V3 Abs, like the CD4i Abs, inhibited gp120 binding to chemokine receptors (Trkola, A et al. (1996) Nature 384:184-87), were found in most HIV+ subjects, and were represented by a plethora of mAbs. In contrast to the CD4i Abs, complete anti-V3 IgG molecules can neutralize. However, long-term antigenic stimulation may be needed to induce anti-V3 Abs with truly broad cross-neutralizing activity, and a significant number of viruses may be resistant to neutralization by typical anti-V3 Abs due to partial epitope masking.

No single category of epitopes points to a direct path for immunogen design. Perhaps the most controversial of these regions as a practical neutralizing target is the V3 loop. Anti-V3 Abs were originally defined as “isolate-specific,” which has become an “accepted misconception” in the field despite extensive demonstrations, published since the mid-1990s, that anti-V3 Abs have much broader cross-reactivity than originally thought.

An explanation for this cross-reactivity is now available: Despite its sequence variability, structurally, V3 is a semi-conserved region subject to stringent constraints given its participation in chemokine receptor binding. Moreover, the ability of anti-V3 Abs to protect against HIV has been well-documented in several animal models. The present inventor and colleagues have shown that individual anti-V3 mAbs are able to neutralize many isolates both within and between clades. The V3 domain thus contains immunogenic, semi-conserved epitopes capable of inducing cross-reactive Abs, and, as such, may serve as a valuable target for HIV vaccines.

Anti-V3 Abs are found in >90% of infected subjects with mean serum levels of ˜80 μg/ml. Interestingly, anti-V3 titers are 10-fold lower than Ab titers to the non-neutralizing immunodominant domain of gp41, suggesting that HIV can divert the immune response to biologically irrelevant targets. Thus, in the context of gp120 and/or the entire virion, the immunogenic potential of V3 appears to be “devalued”, although not abrogated. Indeed, it has been suggested that a significant portion of serum anti-V3 Abs might be induced by viral “debris” present when V3 no longer exists in relevant conformations (Parren et al., supra). In fact, many anti-V3 Abs cannot neutralize primary isolates effectively, so perhaps only a minority of serum anti-V3 Abs are neutralizing. This phenomenon would suggest that few infected subjects have enough circulating anti-V3 (or other protective) Abs to prevent superinfection—a situation that appears to be the case (Chohan, B et al. (2005), J Virol 79:10701-8). A prophylactic immunogen or vaccine, of the type envisioned in the present invention, must “do better than Nature”, i.e., induce protective Abs at higher levels and with broader specificity than occurs during natural infection. As disclosed herein, this may well be achieved if the immune response can be focused on an epitope which induces broadly-reactive neutralizing Abs, such a V3.

As noted above, a potential hurdle to developing effective anti-V3 vaccines is that V3 is at least partially masked (like the other known neutralizing epitopes), at least part of the time, in at least some of the neutralization-resistant isolates. There appears to be no V3 masking in neutralization-sensitive viruses (“Tier 1” viruses (Mascola, J R et al., 2005, J. Virol. 79:10103-10107; see also “Detailed Description” section below). V3 is probably masked in the more resistant viruses by the V1/V2 loop and/or glycans (Wei, X et al. (2003) Nature 422:307). Nonetheless, V3 obviously must be exposed at least transiently in order for it to participate in co-receptor binding.

As noted above, generating anti-HIV-1 neutralizing antibodies remains a major challenge for HIV-1 vaccine development. The present invention exploits the improved assay accuracy and the availability of more standardized reagents and clonal viruses provide to provide immunogens and methods to induce broadly-reactive cross-clade NAbs against HIV and to assess improvements in breadth and potency of neutralization that might not otherwise be appreciated.

### SUMMARY OF THE INVENTION

To enhance the quality and/or quantity of neutralizing Abs in immune sera in furthering the concept of “immunofocusing vaccines” the present inventor developed an immunization regimen designed to focus the immune response on the V3 loop of gp120. To do this, both classical immunologic approaches to priming and selective stimulation memory B cells (Ovary, Z et al. (1963) Feder Proc. 22:2) were used along with more an appreciation of importance of the conformation of B cell epitopes (Gorny, M K et al. (2002) J Virol 76:9035-45).

According to the present inventor's conception, V3 exposure is briefer in resistant viruses, but occurs nonetheless, probably during the conformational change that takes place during the transition from the CD4-unliganded to CD4-liganded form of gp120 (see Chen, B et al. (2005 (Nature 433:834). Thus, neutralization of more resistant viruses may require higher affinity, and/or higher levels of, anti-V3 NAbs. This conception is supported by results described herein. The present inventor and colleagues have also demonstrated the existence of “complex V3 epitopes” composed of regions of V2 and V3, and targeting of these epitopes by human mAbs (Gorny, M K et al. (2005) J Virol. 79:5232-7)

An immunization approach of the present invention that differs from those in the prior art is the construction and use of an immunogen that will focus the immune response on one or a few epitopes that are known to induce anti-HIV neutralizing Abs (Nabs). To test whether this approach enhances the quality and quantity of NAbs in sera of immunized subjects, animals, studies were designed to focus the immune response on the V3 loop of HIV gp120. A number of features made V3 a logical first target in the induction of a focused NAb response. Many studies have shown that anti-V3 Abs can neutralize diverse strains of HIV. As noted above, it has long been known that human mAbs directed against V3 can neutralize primary isolates (e.g., Binley et al., 2004, supra) and that (polyclonal) anti-V3 Abs in the sera of patients (Krachmarov C P et al. (2001), AIDS Res Hum Retrovir 7):1737-48); Krachmarov C P et al., (2005) J Virol. 79:780-90) and immunized guinea pigs and monkeys have neutralizing activity (Liao, H X (2000) J Virol. 74:254-63; Yang X et al. (2004) J Virol. 78:12975-86; Chakrabarti et al., supra). V3 is a highly immunogenic region of the virus envelope (Carrow, E W et al. (1991) AIDS Res Hum Retrovir: 7:831-8; Vogel, T et al. (1994) J Immunol 153:1895-904); it is formed by a continuous (rather than discontinuous) stretch of amino acids.

It has long been known that levels of Abs in humans achieved after immunization can reach several hundred μg/ml of serum (Kabat & Mayer, supra). Thus, one underlying conception of the present invention is that an immunogen, or cocktail of immunogens, can focus the immune response on an epitope that induces Abs to the relevant V3 conformation(s), and that, if not diverted by biologically irrelevant epitopes, such immunogen(s) can “do better than Nature,” inducing biologically effective levels of Abs that will block HIV infection.

As noted above, this cross-reactivity of anti-V3 Abs is counterintuitive if one focuses on sequence variability rather than on the conservation of V3 structures that mediate selection of and interaction with chemokine receptors. This structural conservation of the V3 loop explains the immunochemical cross-reactivity between Abs and V3s of differing sequence; similar cross-reactivity phenomena are well-documented throughout the history of immunochemistry (Kabat & Mayer, supra). Thus, regardless of its sequence variability within and between clades, V3 has common structures required for gp120 interaction with chemokine receptors. Portions of V3 may be targeted by the known cross-reactive V3 mAbs, and according to the present invention, appropriately designed immunogens will induce these and even more extensively cross-reactive anti-V3 Abs that will block the interaction of gp120 with the co-receptors.

Presented herein are examples of studies in rabbits in which new versions of the prime/boost approach were used to preferentially induce broadly-reactive cross-clade anti-V3 Abs. Subjects were primed (as in earlier studies) with one or more gp120 DNA constructs. However, the proteins used as booster immunogens in the prior art, such as recombinant Env proteins or recombinant adenovirus vectors carrying HIV Env, were replaced with immunogenic fusion proteins that present only the V3 region of Env. The results show this approach induces a vigorous Ab response and that the NAbs thus induced, which display cross-clade neutralizing activity, are primarily directed against the V3 region of gp120. The results prove the concept that V3 can induce Abs that recognize multiple V3 loops and that anti-V3 Abs induced by such immunization can mediate cross-clade neutralization. The present invention thus demonstrates that focusing the humoral immune response on a specific neutralization domain, such as V3, is a rational and advantageous approach to vaccine development.

### BRIEF DESCRIPTION OF THE DRAWINGS

FIG. 1. Humoral immune responses of the groups of immunized rabbits described in Tables 2 and 3, and Example I. (Left column) Activity of serum Abs binding in ELISA to MuLV gp70. o, pre-bleed; , two weeks after second protein boost. (Right column) Binding activity of serum drawn two weeks after the second protein boost vs. gp120 core of YU-2 (□) or gp120 core of YU-2 containing V3 (▪). Y-axis represents Absorption (OD); X-axis represents the reciprocal of the serum dilutions, Data shown are from one representative experiment.

FIG. 2. Titration of neutralizing activity against CRF02_AG virus DJ263 in rabbit immune sera drawn two weeks after the second protein boost. Rabbit groups are described in Table 2 and 3. Each panel shows the results from the three animals in each group. The percent neutralization was calculated on the basis of the activity of the immune sera vs. the activity in the pre-bleed sera from a rabbit in the corresponding group.

FIG. 3. Neutralizing activity in immune rabbit sera (at a final dilution of 1:20) against CRF02_AG primary isolate DJ263. Sera from two time points were evaluated: two weeks after the third DNA prime (hatched bars), and two weeks after the second protein boost (solid bars). The percent neutralization was calculated on the basis of the activity in the immune sera vs. the corresponding animal's pre-immune sera. Data shown are from one representative experiment.

FIG. 4. Neutralizing activity against primary isolate DJ263 from CRF011_cpx in immune rabbit sera prior to (hatched bars) or after (solid bars) incubation of sera with 180 μg/ml of a 23-mer peptide representing the V3 consensus sequence from clade B. Data are shown for sera from each of the three rabbits in Groups I-1: -/B, Group I-2: A_{R}/B, and Group I-3: A_{R}/gp¹²⁰_{R}, as defined in Tables 2 & 3.

FIG. 5. Geometric mean titers for 90% neutralization (GMT₉₀) of V3 chimeric pseudoviruses measured in immune rabbit sera obtained two weeks after the second protein boost. Titers are shown at which relative luminescence units (RLUs) were reduced 90% compared to control wells containing virus alone. Data are derived from two to three neutralization assays. Results from the three rabbits in each of the groups described in Table 2 & 3 are shown:
-/B (□), A_{R}/B [symbol] A_{R}/gp120_{R} [symbol] -/ABC [symbol] A_{R}/ABC [symbol] C_{Q}/ABC(▪), A_{R}+C_{Q}/ABC [symbol] A_{R}/B [symbol] Consensus V3 sequences inserted into the SF162 backbone are:

| | | |
|---|---|---|
| B | CTRPNNNTRKSIHIGPGRAFYTTGEIIGDIRQAHC | SEQ ID NO:1 |
| F | ------------H----Q---A--E------K--- | SEQ ID NO:2 |
| AE | ----S----T--T----QV--R--D------K-Y- | SEQ ID NO:3 |
| A1 | ------------R----Q---A--D---------- | SEQ ID NO:4 |
| AG | -----------VR----QT--A--D---------- | SEQ ID NO:5 |
| *C | ------------R----QT--A--D---------- | SEQ ID NO:6 |
| *H | ------------HL---Q---A--D---------- | SEQ ID NO:7 |

(*Experiments not shown included pseudoviruses
carrying the consensus sequences of the indicated
2 clades)

FIG. 6. Geometric mean titers for 50% neutralization (GMT₅₀) of two primary isolates measured in immune rabbit sera obtained two weeks after the second protein boost. Titers are shown at which RLUs were reduced 50% compared to control wells containing virus and pre-immune serum from the corresponding animal. The rabbit groups, as described in Table 2, are denoted in the legend.

FIG. 7. New primer for evoking anti-gp120 cross-clade immunity. gp¹²⁰_{ABC} is a gp120 priming DNA construct carrying 3 moles of V3 for each mole of gp120. The V1V2, V3, and V4 loops in this clade A gp120 molecule were replaced with V3 consensus sequences of clades A, B, and C, respectively.

### DESCRIPTION OF THE PREFERRED EMBODIMENTS

The present invention is based on founding principles of protein structure, immunology and virology. It focuses on ways to produce broadly neutralizing human anti-V3 Abs similar to those induced by the natural infection process by (a) identifying common structural features of a desired/selected HIV-1 epitope(s), such as a V3 epitope that is recognized by these Abs, and (b) designing and producing immunogens (for priming or boosting responses) based on these structures, (c) use of these immunogens as vaccines in animal models and then in patients, and (d) testing of the sera from immunized subjects for broad neutralizing activity.

The present invention differs from previous failed attempts to induce broadly neutralizing Abs by focusing the immune system on a single, neutralizing Env epitope rather than on all Env epitopes, by designing an immunogen for use in boosting the Ab response that retains the native structural conformation of the neutralizing epitope (e.g., the V3 loop) as it appears on the surface of the virus in vivo. While the present inventor initially exemplify their invention using V3, this invention extends logically and directly to other neutralizing epitopes of HIV, and, indeed, to neutralizing epitopes of other pathogens.

The present invention is based on studies that identified how certain DNA immunogen priming followed by HIV-1 epitope fusion protein boosting would focus the humoral immune response on a single neutralizing epitope of HIV-1 (HIV) gp120 and result in the induction of broadly, cross-clade neutralizing antibodies (“NAbs”)

The present invention is directed to an immunogenic composition for boosting a broadly-neutralizing cross-clade anti-HIV antibody response in a subject who has been primed with an immunogen that primes for the antibody response, the composition comprising in unit dosage form one or more HIV-1 neutralizing epitopes each of which is in the form of a fusion protein that includes:
(a) a first fusion partner that comprises a neutralizing epitope of HIV-1 Env protein fused to
(b) a second fusion partner that is a polypeptide which, when fused to the first fusion partner, results in a fusion protein that adopts a conformation of the epitope that promotes an antibody response specific for the epitope upon immunization of a subject with the composition,
   wherein administration to a primed subject of
   (i) one unit dose of the immunogen, or
   (ii) more than one unit dose of the immunogen simultaneously at different sites and/or sequentially,
      results in a boosted broadly neutralizing cross-clade anti HIV-1 antibody response in which a serum neutralizing antibody titer is increased at least 4-fold (or alternatively, is increased by at least 3 standard deviations above the mean) against at least two Tier 1 primary isolates (defined below) from at least two different HIV-1 clades compared to the neutralizing titer (or mean neutralizing titer) of serum from similarly primed but non-boosted subjects.

The epitope in this fusion protein is not necessarily a consensus sequence or even a sequence that exists in nature (i.e., not found in any Env sequence examined thus far) but is an epitope that will induce the desired broad NAb response. Thus, the epitope may be linear or result from discontinuous regions of the Env protein. The epitope (e.g., a V3 epitope; see below) should be in its correct conformation as it is found on the viral envelope, i.e., glycosylated, disulfide linked, etc.

In the above composition, the administration to the primed subject may result in a serum neutralizing antibody titer of at least 1:20 against Tier 1 primary isolates.

In other embodiments, the number of Tier 1 primary isolates against which the NAb response is measured may be at least 3, at least 6, at least 12, etc.

In the above composition, the unit dosage is preferably between about 20 and 200 μg of the boosting immunogen. Preferably the number of unit doses of the boosting immunogen given to result in the boosted neutralizing titer as above results in a cumulative administered dose of about 100 μg to about 200 μg of the boosting immunogen.

In one embodiment of the above composition, the first fusion partner comprises more than one neutralizing epitope of the Env protein.

In the above composition, when the epitope is one that has a variable amino acid sequence among HIV-1 isolates in a clade, the first fusion partner may have the amino acid sequence that is a consensus sequence of the epitope from a single clade of HIV-1 viruses.

In one embodiment of the above composition, (A) the first fusion partner epitope has an amino acid sequence of a clade A, B or C virus, or (B) the first fusion partner comprises more than one neutralizing epitope, each of which has an amino acid sequence of a clade A, B or C virus.

In another embodiment of the above composition the amino acid sequence of the first fusion partner epitope or epitopes is a consensus sequence of the epitope from a clade A, B or C virus.

In the above composition the neutralizing epitope is preferably a V3 epitope and the fusion protein comprises the V3 epitope. In other embodiments, the epitope may be a CD4 binding domain/site (CD4bs) epitope or a CD4-induced (CD4i) epitope, etc. The fusion protein may include two or more of a single epitope or a mixture of different epitopes. The V3 epitope of the fusion protein may comprise the amino acid sequence GPGR (SEQ ID NO:17) or GPGQ (SEQ ID NO:18).

Thus, the boosting immunogen composition may include a mixture of two or all of:
(i) the fusion protein combines in which the first fusion partner has the amino acid sequence of V3 of a clade A virus or the consensus V3 sequence of clade A viruses;
(ii) the fusion protein in which the first fusion partner has the amino acid sequence of V3 of a clade B virus or the consensus V3 sequence of clade B viruses;
(iii) the fusion protein in which the first fusion partner has the amino acid sequence of V3 of a clade C virus or the consensus V3 sequence of clade C viruses.

In the above composition, the second fusion partner may be MuLV gp70, as exemplified herein. However, more generally, the fusion protein boosting immunogen may include one or more epitopes inserted into a fusion protein that can assemble into oligomers in which the epitope would be exposed to the immune system. One example is the immunoglobulin (Ig) molecule in which the a IgH chain fusion protein and a Ig L chain fusion protein each comprise one or more desired HIV epitopes, and then assemble into a dimer (IgG-like) or pentamer (IgM-like) that present two or five (in this example) “copies” of the epitope(s). Other examples of a preferred fusion partner are mucin and the soybean-derived Bowman-Birk trypsin inhibitor.

The present invention is also directed to a composition that comprises both a priming immunogen and a boosting immunogen.

In a preferred embodiment, such an immunogenic composition for both priming and boosting a broadly-neutralizing, cross-clade anti-HIV-1 antibody response specific for a selected HIV-1 neutralizing peptide epitope, comprises:
(a) a specific priming immunogen for the peptide epitope in unit dosage form that comprises DNA encoding an HIV-1 polypeptide in which an amino acid sequence of the epitope is present; and
(b) in unit dosage form, a specific boosting immunogen specific for the epitope, which boosting immunogen is that described above, namely a fusion protein that includes:
   (i) a first fusion partner that comprises a neutralizing epitope of HIV-1 peptide Env protein fused to
   (ii) a second fusion partner that, when fused to the first fusion partner, results in a fusion protein that adopts a conformation of the epitope that promotes an antibody response specific for the epitope upon administration to a subject that has been primed with the priming immunogen.
      The above composition may be further is characterized as follows:
(1) priming of a subject with one or more unit doses of the priming immunogen, followed by
(2) boosting the subject with
   (i) one unit dose of the boosting immunogen or
   (ii) more than one unit dose of the immunogen simultaneously at different sites and/or sequentially
      results in a boosted broadly neutralizing cross-clade anti HIV-1 antibody response in which a serum neutralizing antibody titer is increased at least 4-fold (or at least 3 standard deviations) against at least two Tier 1 primary isolates from at least two different HIV-1 clades compared to the neutralizing titer of serum from either similarly primed but non-boosted subjects, or unprimed but similarly boosted subjects.

In the above composition the unit dosage of the boosting immunogen is preferably between about 20 and 200 μg of the fusion protein, and the number of unit doses of the boosting immunogen required to yield the boosted neutralizing titer defined as above results in a cumulative administered dose of about 100 μg to about 200 μg of the boosting immunogen.

In the above composition the unit dosage of the priming immunogen is about 1 μg to about 100 μg of the DNA, and the number of unit doses of the priming immunogen given to achieve the boosted response results in a cumulative administered dose of about 20 μg to about 100 μg of the DNA.

All of the embodiments of the boosting immunogen may be used as the boosting component of the above “priming plus boosting” composition. Thus, if the first fusion partner of the fusion protein may have an amino acid sequence of a clade A, B or C virus or a consensus sequence of the epitope from a clade A, B or C virus. The neutralizing epitope is preferably a V3 epitope and the boosting immunogen may optionally comprise a combination of V3 fusion proteins or a V3 fusion protein that includes two or more of the same or different V3 epitopes as indicated above.

In the above composition, the priming immunogen may comprise
(A) env DNA encoding an Env protein bearing an amino acid sequence of GPGR (SEQ ID NO:17) corresponding to the tip of the V3 peptide loop, and/or
(B) env DNA encoding an Env protein bearing an amino acid sequence of GPGQ (SEQ ID NO:18) corresponding to the tip of the V3 peptide loop.

In the above priming+boosting composition, the V3 fusion protein combination may be a mixture of two or all of:
(i) a fusion protein in which the first fusion partner has the amino acid sequence of V3 of a clade A virus or the consensus V3 sequence of clade A viruses;
(ii) a fusion protein in which the first fusion partner has the amino acid sequence of V3 of a clade B virus or the consensus V3 sequence of clade B viruses;
(iii) a fusion protein in which the first fusion partner has the amino acid sequence of V3 of a clade C virus or the consensus V3 sequence of clade C viruses.

The second fusion partner in the boosting immunogen may be MuLV gp70 or other polypeptides as described above.

The present invention also provides an immunogenic pharmaceutical composition comprising the above immunogenic composition and an immunologically and pharmaceutically acceptable carrier or excipient; examples of such carriers or excipients are well-known in the art.

The present invention also includes a kit comprising in separate compartments in close proximity therein:
(a) one or more unit dosages of the boosting immunogenic composition as above, and
(b instructions for administering the boosting immunogenic composition to a subject for boosting the antibody response.
   her kit comprises, in separate compartments in close proximity therein:
one or more unit dosages of the priming immunogen as above;
one or more unit dosages of the boosting immunogen as above; and
instructions for administering the priming and the boosting immunogens to a subject for producing the antibody response.

The above kit may further comprises an adjuvant or immunostimulatory protein different from the fusion protein, and instructions for administering the adjuvant or immunostimulatory protein.

Also provided in this invention is a method of immunizing a mammalian subject, preferably a human, to produce a broadly-neutralizing cross-clade anti-HIV antibody response specific for an HIV-1 neutralizing epitope, comprising administering, to a subject who has been primed with an immunogen that primes for the antibody response, one or more unit doses of an immunogenically-effective amount of the immunogenic booster composition or pharmaceutical composition as above, wherein the immunization results in a boosted broadly neutralizing cross-clade anti HIV-1 antibody response in which a serum neutralizing antibody titer in the subject is increased at least 4-fold (or at least 3 standard deviations) against at least two Tier 1 primary isolates each from at least two different HIV-1 clades compared to the neutralizing titer of serum from similarly primed but non-boosted subjects. Preferably, the method results in a serum neutralizing antibody titer of at least 1:20 against the Tier 1 primary isolates.

Also provided is a method of immunizing a mammalian subject to produce a broadly-neutralizing cross-clade anti-HIV antibody response specific for an HIV-1 neutralizing epitope, comprising administering to a subject, preferably a human, an effective immunogenic amount of the priming+boosting composition as above or the pharmaceutical composition thereof. The method comprises
(a) priming the subject with one or more unit doses of the priming immunogen; and
(b) between about one and about 12 weeks after the priming, boosting the subject with one or more simultaneous or sequential unit doses of an immunogenically effective amount of the boosting immunogen,
   wherein the immunization results in a boosted, broadly neutralizing cross-clade anti HIV-1 antibody response in which a serum neutralizing antibody titer in the subject is increased at least 4-fold (or increased at least 3 standard deviations) against at least two Tier 1 primary isolates each from at least two different HIV-1 clades compared to the neutralizing titer of serum from either similarly primed but non-boosted subjects, or unprimed but similarly boosted subjects.

The method preferably results in a serum neutralizing antibody titer of at least 1:20 against the Tier 1 primary isolates.

The method as described above may further comprise administering an adjuvant or an immunostimulatory protein different from the fusion protein, such as a cytokine, before, during, or after the priming or the boosting. Preferred adjuvants include (a) ISAF-1 (5% squalene, 2.5% pluronic L121, 0.2% Tween 80) in phosphate-buffered solution with 0.4 mg of threonyl-muramyl dipeptide; (b) de-oiled lecithin dissolved in an oil (e.g., AMPHIGEN™ (c) aluminum hydroxide gel; (d) a mixture of (b) and (c); (e) QS-21; (f) monophosphoryl lipid A adjuvant. In the case of certain mammals, a preferred adjuvant is incomplete Freund's adjuvant.

In the present method, the boosting immunogen is preferably administered intradermally, subcutaneously or intramuscularly. The priming immunogen is preferably administered by needle-less jet injection (biolistic injection), intradermal injection, intramuscular injection, epidermal patch, epidermal abrasion, or gene gun delivery (intramuscular, intradermal or both).

The mammalian subject in the present methods may be a rodent, a rabbit, a non-human primate, or a human. In the case of humans, the subject may be one who is susceptible to, or at risk of, HIV-1 infection, or a subject infected with HIV-1.

The inventor has used an animal model in which rabbits were immunized with three priming doses of gp120 DNA plasmids derived from HIV env genes from a virus carrying a clade A Env bearing the GPGR motif (SEQ ID NO:17) at the tip of the V3 and/or from a virus carrying a clade C Env bearing the GPGQ motif at the tip of the V3 loop. The rabbits subsequently received two booster immunizations with recombinant fusion proteins (FPs) consisting of a truncated form of the MuLV gp70 Env protein (as a “carrier”) and the consensus V3 sequence (V3-FPs) from either HIV clades A, B or C (V3A-FP, V3B-FP and V3C-FP, respectively). Immune sera from subjects receiving various prime/boost regimens neutralized primary isolates from strains heterologous to those from which the immunogens were constructed. 50% neutralizing titers against primary isolates from clade B, CRF01_AG and CRF-11_cpx ranged from 1:46 to 1:559. Neutralizing activity was primarily due to V3-specific antibodies as shown by peptide absorption studies. Sera were also tested for activity against pseudoviruses carrying the SF162 env in which the native V3 region was replaced with the consensus V3 regions from several clades. The V3 loop in the SF162 Env exists in an unmasked form so that these pseudoviruses are extremely sensitive to neutralization permitting the calculation of 90% neutralization titers.

Cross-clade NAbs were demonstrated against the V3 chimeric pseudoviruses carrying the consensus V3 sequences from clades A1, AG, B, AE, and F. Neutralizing Ab levels after the V3-FP boosts were generally better than those elicited with two gp120 boosts. The broadest neutralizing activity was elicited using as a priming immunogen gp120 DNA from clade C virus and as boosting immunogens, a combination of V3-FPs carrying V3 sequences from clades A, B and C. Thus, the inventor discovered that cross-clade HIV neutralizing antibodies could be elicited by immunofocusing the Ab response on a neutralizing epitope such as V3.

Immunofocusing

The term “immunofocusing” as used herein means intended a process of inducing an immune response, preferably an Ab response, the includes priming and boosting, although primarily is concerned with the boosting phase. An immunofocused response is one in which the stimulation, particularly in the boosting phase is done using an immunogenic form of the desired epitope, e.g., a V3 epitope, to induce neutralizing/protective Abs by designing or selecting the boosting immunogen as described herein to focus the immune system on the epitope of interest. This may be accomplished by removing or limiting the presence of undesired or irrelevant or competing epitopes from the boosting immunogen, for example, by using a fusion protein between a particular V3 epitope, for example, and a fusion partner (that can be considered a carrier) rather than a full Env protein that includes a multitude of additional HIV epitopes (from the V3 region and non-V3 epitopes). Use of such a boosting immunogen will stimulate a primed immune system to focus on the selected epitope(s) that will result in higher titer NAbs with the desired properties of broad reactivity and cross-clade neutralizing activity.

Others have used the term “immunofocusing differently, for example, R Pantophlet and D R. Burton (2003) “Immunofocusing: antigen promote the induction neutralizing antibodies,” Trends Mol Med. 9:468-73. This document did not really define the term. However the distinction from the present use of “immunofocusing” is evident. Pantophlet and Burton stated that their studies using monomeric gp120 as antigen provided additional support for an approach they termed “immunofocusing.” Their goal was to formulate immunogens that would induce Abs with neutralizing properties equivalent to those of a particular broadly neutralizing mAb b12 (which defines an epitope “b12”). To this end, they constructed a hyperglycosylated gp120 mutant containing 7 additional N-glycosylation motifs at specific sites, plus four Ala substitutions in the Phe⁴³ cavity. These modifications abolished the binding of a panel of non-neutralizing gp120 Abs to as well as a polyclonal antiserum of low neutralizing potency. The mutant retained b12 binding, albeit at reduced affinity. (They went on to improve b12-binding affinity by modifying this mutant further by reverting one of the added glycosylation motifs back to wild-type and showed too that by removing N-terminal residues in the mutant gp120, they eliminated binding of three non-neutralizing mAbs (that had bound the original hyperglycosylated mutant). Thus, they generated a panel of antigens that they believed could be advantageous in directing the Ab response effectively towards the b12 epitope.

### Measurement of Neutralization of HIV and Standardization of Protocols

For optimal evaluation and comparison of vaccine immunogens, a preferred embodiment of the present invention makes use of DNA plasmids encoding full-length functional Env proteins. These env clones, when transfected along with an HIV-1 env defective molecular clone, produce well-characterized HIV Env pseudovirions (PsV's). Additionally, standardized panels of Env-pseudotyped viruses are used to assess the potencies and breadths of NAbs elicited by the immunogens being tested. These virus panels are preferably also used in neutralization assays that evaluate sera from clinical immunization studies as well as in the preclinical evaluation of vaccine immunogens.

A number of assays are used in the art to measure antibodies that neutralize HIV-1 (and the related simian immunodeficiency virus (SIV) and simian/human immunodeficiency virus (SHIV) (Mascola, et al., supra, and reference cited therein, all of which are hereby incorporated by reference. While relying on different technologies, these assays are based on the principle of measuring reductions in virus infectivity in cells that express the suitable fusion receptors for virus entry. See Table 1, below). These assays can differ with regard to:

(1) the type of target cells (e.g., neoplastic T-cell lines, primary human lymphocytes, or genetically engineered cell lines),

(2) the methodology for detecting viral infection (e.g., p24 antigen, reverse transcriptase (RT), cell killing, plaque formation, or reporter gene expression),

(3) the type of virus used, and whether single or multiple rounds of infection are permitted (e.g., uncloned PBMC-derived stocks, uncloned or molecularly cloned PsV, or replication-competent chimeric molecular clones), and

(4) whether single or multiple rounds of infection are permitted (e.g., Env-pseudotyped viruses produce a single round of target cell infection).

The plasmid expression vectors used to provide Env in trans can be clonal or can contain a quasispecies of env genes derived from a patient sample.

TABLE 1
Common assays used to measure neutralizing antibodies against HIV-1.

| HIV | Target Cells | Measure of Infection |
|---|---|---|
| T-cell line adapted | Neoplastic CD4+ T cell | Syncytia or plaques |
| | line expressing CXCR4 | Cell killing |
| | | Gag antigen expression |
| Primary Isolates | Primary human T cells | Gag antigen expression |
| | | RT activity |
| Primary isolates, | Genetically engineered | Luciferase |
| Env pseudoviruses, | cell lines expressing | Green fluorescent protein |
| chimeric infectious | CD4, CCR5 and CXCR4 | Secreted alkaline |
| molecular clones | | phosphatase |
| | | β-galactosidase |

While these diverse assays can produce qualitatively similar results in terms of how each assay rank-orders neutralization potency (6, 40), they may differ in accuracy and reproducibility and adaptability to large sets of samples.

Viral diversity has been an major obstacle for effective Ab-based immunization against HIV-1. According to the present invention, an effective, an HIV immunogen/vaccine is one that generates antibodies that neutralize a genetically and antigenically diverse set of viruses. Thus, to ascertain the breadth of NAb responses in a meaningful way, use of multiple viral strains are preferred in neutralization assays. Commonly, different laboratories use different HIV-1 strains, which contributes to a lack of uniformity that has made comparison of immunogens difficult. Thus, there is a pressing need to establish standard panels of HIV-1 strains for wide distribution and use. The creation of standard virus panels would facilitate proficiency testing and GLP assay validation and would allow consistent data sets to be acquired that could be used to compare new immunogens and to prioritize the advancement of candidate vaccines. This prioritization could occur at the preclinical stage, to decide which vaccines to test in humans, and during phase I/II trials, to prioritize candidate vaccines for advanced clinical development. Standard panels would also allow refined measurements that might reveal incremental improvements in immunogen design. This would provide an increased understanding of the barriers to effective NAb induction and identify vaccine design concepts that deserve further development.

The use of virus panels described here relates mainly to preclinical testing of candidate immunogens. Thos in the art are still seeking to optimize valid virus panel size by testing whether results obtained with an existing virus panel are predictive of results obtained with a much larger number of strains matched in genetic subtype to the standard panel. According to this invention, a panel of HIV isolates used to assess the breadth of a NAb response is 2 viruses, each from a different genetic subtype or clade, preferably 2 viruses per clade, more preferably 3 viruses per clade, and may include, 4, 5, 6, 7, 8, 9, 10 11 or 12 isolates per clade.

As described by Mascola et al., supra, a systematic approach to the evaluation of NAb responses may be achieved using a three-tier algorithm for evaluating novel immunogens as set forth below.

TIER 1
Neutralization sensitive viruses that are typically not included in the immunogen

TIER 2
Panel of heterologous viruses matching the genetic subtypes (clades) of the immunogen (e.g. 12 viruses per panel

TIER 3
Multi-clade panel comprising six TIER 2 viruses of each genetic subtype, excluding genetic subtype(s) evaluated in TIER 2. May include additional strains geographically important at site of vaccine trials

The present invention is primarily concerned with the use of TIER 1 viruses at the stage of identifying immunogens that elicit at least the indicated minimal level and breadth of HIV NAbs. Sera from recipients of the immunogens of the present invention immunized according to the method described herein would are against homologous virus strains represented in the vaccine and a small number of heterologous viruses that are known to be highly sensitive to Ab-mediated neutralization. Examples of the latter viruses include the primary isolate SF162 and T-cell-line-adapted viruses. According to the present invention, a preferred boosting immunogen (or a preferred method of boosting a primed subject) is one where administration of one or more unit doses of the immunogen results in a boosted broadly neutralizing cross-clade anti HIV-1 Ab response in which a serum neutralizing Ab titer is increased at least 4-fold against at least 2 TIER 1 primary isolates from at least two different HIV-1 clades compared to the neutralizing titer of serum from similarly primed but non-boosted subjects. In another embodiment, the titer is increased at least by the amount indicated in at least about 3 or 4 or 5 or 6 or 7 or 8 or 9 or 10 or 11 or 12 Tier 1 primary isolates.

Testing in TIER 2 and TIER 3 would provide a greater measure of neutralization breadth when comparing immunogens. TIER 2 would, for example, utilize the virus panels of 12 viruses from each major clade/genetic subtype (A, B, C, D, E, and A/G), to test neutralizing activity against viruses that are matched in genetic subtype to the immunogen strain. For example, in a TIER 2 test, an Env immunogen based on a virus strain from clade C would be tested against the clade C virus panel. This immunogen could be compared to other immunogens designed to elicit clade C NAbs.

To assess breadth of neutralization against viruses from other clades, a TIER 3 virus panel would, for example, consist of a total of six viruses from each of the heterotypic clades (i.e., in the case of a clade C immunogen, TIER 3 would include six viruses each from clades A, B, D, E, and A/G). TIER 3 testing may also include an additional set of viruses from the specific region of the world where the immunogen is to be tested. TIER 3 testing would be conducted after neutralization against TIER 2 viruses was detected.

At the present time, only limited numbers of HIV-1 strains that meet the criteria for selection as discussed above are available as candidates for inclusion in standard panels. The genetic and phenotypic characterization of an initial panel of well-characterized molecularly cloned pseudoviruses for clade B has been completed, and the Env expression plasmids corresponding to an initial panel of well-characterized molecularly cloned pseudoviruses for clade B are now available through the NIH AIDS Research and Reference Reagent Program (Li, M et al. (2005) J. Virol. 79:10108-125). Others that can be used are, for example, based on well-characterized multiclade isolates from chronically infected individuals (Brown, B K et al. (2005) J. Virol. 79:6089-6101) and other isolates that may be obtained from the NIH AIDS Research and Reference Reagent Program (www.aidsreagent.org).

In summary, immunological assessment HIV immunogens according to the present invention are preferably tested against standardized panels of pseudoviruses to allow comparisons of the potencies and breadths of elicited NAbs.

### Routes and Schedules of Immunization

DNA Immunization (Priming)

Priming of Ab responses, preferably of IgG Ab responses, with a DNA immunogen is performed using any of a number of routes know in the art. One preferred route is intradermal (ID) gene gun immunization in which DNA-coated gold particles in an effective amount are delivered using a helium-driven gene gun (BioRad, Hercules, Calif.) with a discharge pressure set at a known level, e.g., of 400 p.s.i.

The DNA immunogen may be administered by needle-free jet (biojector) such as the Biojector 2000 (Bioject Inc., Portland, Oreg.) which is an injection device consisting of an injector and a disposable syringe. The orifice size controls the depth of penetration. For example, 50 μg of DNA may be delivered using the Biojector with no. 2 syringe nozzle. Biojector administration is typically via subcutaneous (SC), IM or both SC and IM routes.

Other modes of administration are ID, intramuscular (IM) or subcutaneous (SC) injection (or a combination) using a conventional syringe needle; by epidermal patch or by epidermal abrasion.

There is substantial knowledge in the art (discussed below) as to various routes and modes of immunization with DNA vaccines; it is known in the art that certain routes may be more effective than others, but that multiple routes result in immunogenic effects. It is within the skill of the art to determine, without undue experimentation, whether a particular route works best in the present methods. would be best for priming the desired Ab responses. Pardoll, D et al., Immunity 3:165-9 (1995) that focused on naked DNA vaccines, and discuss, inter alia, involvement of antigen presenting cells (APCs) in the milieu of IM vaccination. As discussed in this reference, and as is well-known in the art, bone-marrow-derived antigen presenting cells (APCs) which are important targets for DNA (or other) vaccines are found in many sites of the body including the skin and muscle tissue, as well as all lymphoid tissues and organs, blood, and in numerous other locations. Skin is one site where APCs have been well-studied. Moreover, this reference shows inflammatory changes in muscle into which a DNA vaccine preparation had been administered. Comparative tests of alternative modes of immunizing mice with an immunogenic plasmid DNA molecule that encodes an antigen linked to HSP70 (Trimble, C et al., 2003, Vaccine 21:4036-42) showed that the most efficacious route was via gene gun compared to needle IM and biojector administrations. However these other routes also generated immunity. Gene gun administration is primarily an ID route, while biojector administration involves either subcutaneous (SC) or both SC and IM routes. Lemon S M et al. J Med Virol 1983; 12:129-36, assessed the feasibility of SC jet injection of a DNA vaccine for hepatitis B and showed this route to be safe and immunogenic, approximating that associated with IM needle injection. Aguiar J C et al., Vaccine 2001, 20:275-80, compared a needle-free Biojector device with syringe/needle for administering a DNA malaria vaccine to rabbits. They examined animals injected by the IM route using a syringe/needle combination, a second group IM with the Biojector device and a third group both IM and ID using the Biojector. While all routes resulted in immune responses, the Biojector IM or IM/ID routes showed greater immunogenicity as compared to the syringe/needle TM route. Rogers W O et al., Infect Immun 2001; 69:5565-72. studied a malaria DNA vaccine in monkeys who received three doses of a mixture of four DNA vaccine plasmids (and a plasmid encoding rhesus granulocyte-monocyte colony- stimulating factor) by various routes (IM by needle injection, IM with the Biojector, or a combination of IM/ID routes by Biojector. Animals immunized by all these routes developed antibody responses against the relevant antigens. The immunized monkeys were either completely or partially protected against challenge with malaria organisms. Bohm W et al., Vaccine, 1998, 16:949-54, studied the induction of humoral and MHC class-T-restricted CTL responses of mice to the small hepatitis B surface antigen (HBsAg) with either a protein antigen or a DNA vaccine. Different routes were used to deliver the HBsAg-encoding plasmid DNA (or the recombinant HBsAg particles): IM, SC. Intraperitoneal (IP), or intravenous. At different time points HBsAg specific antibodies and specific CD8+ T cells were monitored; results showed that IM and SC but not IV nor IP injection of naked DNA efficiently and reliably primed humoral immune responses. Hasan U A et al., Vaccine, 2000, 18:1506-14, evaluated a plasmid vaccine (encoding varicella-zoster virus (VZV) transmembrane glycoprotein gE) in mice. IM and SC injection of VZV gE DNA (without the use of costimulatory molecules or other adjuvant materials) resulted in the generation of antigen-specific antibody responses.

The present DNA constructs are immunogenic when used to prime rabbits and are expected to be immunogenic in humans. DNA immunization (priming) with gp120 constructs result in an effective immune response against a selected Env epitope (preferably focused on V3) from homologous and some heterologous strains of HIV-1 after boosting with the boosting immunogens of this invention. The unit dosage of the priming immunogen is preferably about 1 μg to about 100 μg DNA and the number of unit doses of the priming immunogen results in a cumulative total administered dose of between about 20 μg and about 100 μg DNA.

Preferably, one of more unit doses of the priming immunogen are given at one, two or three time points, preferably separated by between about 2 and 6 weeks, more preferably 2 weeks.

Polypeptide Immunization (Boosting)

The boosting immunogen, preferably one or more fusion proteins as described herein augments the Ab responses to peak levels in subjects already primed with the present DNA priming compositions and methods. The boosting may be by any route known in the art to be immunogenic for proteins, and preferably is via subcutaneously, intramuscular or intradermal administration, or a combination. The boosting immunogen may be administered as one unit dose or, preferably as more than one unit dose given either simultaneously at different sites of the body and/or sequentially over a period of time that may be determined empirically for a given immunogen. Preferably the unit dosage of the boosting immunogen is between about 20 and 200 μg of the fusion protein and the number of unit doses of the boosting immunogen given to result in the desired level of neutralizing titer is a cumulative administered dose of between about 20 μg and 500 μg of the fusion protein, preferably between about 100 μg and about 200 μg.

Preferably, one or more unit doses of the boosting immunogen are given at one, two or three time points. The optimal number and timing of boosts can readily be determined using routine experimentation. Two boosts are preferred. Preferably these boosts are separated by 2 weeks, preferably by 4 weeks, and in other embodiments, 5, 6, 8, 12 weeks, etc., as needed to achieve and maintain the desired titers and breadth of NAbs. It is common that the Ab response remains at relatively high levels for more than 8 weeks after the last boost.

The immunogenic composition of this invention may further comprise one or more adjuvants or immunostimulating agents—which are preferably added to the fusion protein immunogens using for boosting the immune response. An adjuvant is any substance that can be added to an immunogen or to a vaccine formulation to enhance the immune-stimulating properties of the immunogenic moiety, such as a protein or polypeptide. Liposomes are also considered to be adjuvants. See, for example, Gregoriades, G. et al., Immunological Adjuvants and Vaccines, Plenum Press, New York, 1989; Michalek, S. M. et al., Liposomes as Oral Adjuvants, Curr. Top. Microbiol. Immunol. 146:51-58 (1989). Examples of adjuvants or agents that may add to the effectiveness of V3 DNA or polypeptides/peptides as immunogens include aluminum hydroxide, aluminum phosphate, aluminum potassium sulfate (alum), beryllium sulfate, silica, kaolin, carbon, water-in-oil emulsions, and oil-in-water emulsions. Other adjuvants are muramyl dipeptide (MDP) and various MDP derivatives and formulations, e.g., N-acetyl-D-glucosaminyl-(β1-4)-N-acetylmuramyl-L-alanyl-D-isoglutamine (GMDP) (Hornung, R L et al., Ther Immunol 1995 2:7-14) or ISAF-1 (5% squalene, 2.5% pluronic L121, 0.2% Tween 80 in phosphate-buffered solution with 0.4 mg of threonyl-muramyl dipeptide; see Kwak, L W et al., (1992) N. Engl. J. Med., 327: 1209-1238) and monophosphoryl lipid A adjuvant solubilized in 0.02% triethanolamine. Other useful adjuvants are, or are based on, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives such as QS21 (White, A. C. et al. (1991) Adv. Exp. Med. Biol., 303:207-210) which is now in use in the clinic (Helling, F et al. (1995) Cancer Res., 55:2783-2788; Davis, T A et al. (1997) Blood, 90: 509A (abstr.)), levamisole, DEAE-dextran, blocked copolymers or other synthetic adjuvants. Examples of commercially available adjuvants include (a) Amphigen®, an oil-in-water adjuvant made of de-oiled lecithin dissolved in an oil (see for example, U.S. Pat. No. 5,084,269 and US Pat Publication 20050058667A1 and (b) Alhydrogel® which is an aluminum hydroxide gel. Aluminum is approved for human use. Adjuvants are available commercially from various sources, for example, Merck Adjuvant 65® (Merck and Company, Inc., Rahway, N.J.). The immunogenic material may be adsorbed to or conjugated to beads such as latex or gold beads, ISCOMs, and the like.

The immunogenic composition may also be supplemented with an immunostimulatory cytokine, lymphokine or chemokine. Preferred cytokines are GM-CSF (granulocyte-macrophage colony stimulating factor), interleukin 1, interleukin 2, interleukin 12, interleukin 18 or interferon-γ.

General methods to prepare immunogenic pharmaceutical compositions and vaccines are described in Remington's Pharmaceutical Science; Mack Publishing Company Easton, Pa. (latest edition).

Having now generally described the invention, the same will be more readily understood through reference to the following examples which are provided by way of illustration, and are not intended to be limiting of the present invention, unless specified.

### EXAMPLE I

### Materials and Methods

### Construction of Codon Optimized HIV Env DNA Vaccine Constructs

The codon usage of env genes from HIV clade A primary isolate CA1 and clade C 92BR025 (C1) were analyzed with the MacVector software 6.3 against codon preference of Homo sapiens. The codons in CA1 and C1 env genes that are less preferred in mammalian cells were changed to the preferred codons in mammalian systems to promote higher expression of the Env proteins. The codon optimization strategy was not limited to changes of codons for mammalian usage. Sequence optimization was also performed to make the mRNA more stable and the gene more favorable for transcriptional and translational process. During the sequence optimization, the following cis-acting sequence motifs were avoided: internal TATA-boxes, chi-sites and ribosomal entry sites; AT-rich or GC-rich sequence stretches; ARE, INS, CRS sequence elements; cryptic splice donor and acceptor sites; and branch points. Despite such DNA level sequence changes, the final codon optimized CA1 and C1 Env DNA sequences will still produce the same Env amino acid sequences as in the parental HIV-1 primary isolates. These codon optimized env genes were chemically synthesized by Geneart (Regensburg, Germany).

To make codon optimized CA1 and C1 gp120 DNA vaccines, the codon optimized gp120 gene inserts were first PCR amplified from the codon optimized CA1 or C1 env gene. A pair of primers gp120.CA1-opt1 (5′ GTCGCTCCGCTAGCCTGTGGGTGACCGTG 3′ SEQ ID NO:8) and gp120.CA1-opt2 (5′ ACCTACGGATCCTTACTGCACCACTCTTCTCTTGGC 3′, SEQ ID NO:9) were used to amplify the codon optimized gp120 CA1 gene insert. The priming constructs, tp120.Syn-7 (5′ GTCGCTCCAGCTAGCCTGTGGGTGACCGTGTACTACGGC 3′, SEQ ID NO:10) and gp120.Syn-10 (5′ CGACGGATCCTTACTCCACCACGCGGCGCTTGGC 3′, SEQ ID NO:11) were used to amplify the codon optimized gp120 C1 gene insert. Then, the optimized CA1 or C1 gp120 gene insert was cloned into DNA vaccine vector pJW4303 (Wang et al., 2005, supra) at the NheI and BamHI sites downstream of a human tissue plasminogen activator (tPA) leader sequence substituting the natural HA leader sequence. The DNA vaccine plasmids were prepared from Escherichia coli (HB101 strain) with a Mega purification kit (Qiagen, Valencia, Calif.) for both in vitro transfection and in vivo animal immunization studies.

### Protein Immunogens

The V3-fusion proteins (V3-FPs) contained a 45-amino-acid domain of gp120 encompassing the V3 sequences of either JR-CSF (clade B), 92UG037.08 (clade A) or 93IN904 (clade C) (see Table 2). The V3 regions were joined to the C-terminus of a 263 amino acid fragment of the Friend murine leukemia virus (MuLV) gp70, as described (Kayman, S C et al. (1994) J Virol. 68:400-10). To facilitate purification, the His-8 and Gln-9 of the gp70 protein were replaced with a sequence of six His residues (His tag). The V3 fragments in the fusion proteins contain the disulfide-bonded loop and three sites for N-linked glycosylation, one within the V3 loop and one on each flank.

The clade B fusion protein (V3_{B}-FP) was expressed in Chinese hamster ovary (CHO) cells from a glutamine synthetase vector, pEE14 (CellTech, Cambridge, UK), containing the human cytomegalovirus major immediate-early (HCMV MIE) promoter (Kayman et al., supra). Similar clade A and clade C fusion proteins (V3_{A}- and V3_{C}-FPs) were cloned into pcDNA3.1zeo(−) (Invitrogen) and expressed in CHO cells Krachmarov et al., 2005, supra). All fusion proteins were purified on Nickel-nitrilotriacetic acid resin (NTA Superflow; Qiagen, Valencia, Calif.) as described by Krachmarov et al., 2001, supra.

### Immunization Protocol

Female New Zealand White (NZW) rabbits 6-8 weeks old (body weight of ˜2 kg) were purchased from Millbrook Farm (Amherst, Mass.) and housed in the animal facility managed by the Department of Animal Medicine at the Univ. of Massachusetts Medical School in accordance with IACUC approved protocol.

Groups of rabbits were primed with three DNA immunizations at weeks 0, 2, and 4 by a Bio-Rad Helios gene gun (Bio-Rad, Hercules, Calif.). The gp120 DNA vaccine plasmids or the negative control pJW4303 vector plasmid were coated onto 1.0 μm gold beads at a ratio of 2 μg DNA/mg gold. Each gene gun shot delivered 1 μg of DNA and a total of 36 non-overlapping shots were delivered to each rabbit on shaved abdominal skin at each immunization.

The animals then received two boosts with recombinant gp120 JR-FL protein (obtained from the NIH AIDS Research and Reference Reagent Program, catalog no. 4598) or one or more of the V3-fusion proteins at weeks 10 and 14. A total of 100 μg recombinant gp120 protein or of V3-FP(s) was administered intramuscularly with incomplete Freund's adjuvant (IFA) per injection. Blood was collected prior to immunization and two weeks after each immunization.

### Measurement of Ab Levels by ELISA.

To determine reactivity in sera from immunized animals, affinity-purified mammalian-expressed YU2 gp120 core and YU2 gp120 core+V3 was used (provided by Drs. M. Tang and R. Wyatt. The YU2 V3 sequence is CTRPNNNTRKSINIGPGRALYTTGEIIGDIRQAHC [SEQ ID NO:1]. The V3_{A}-, V3_{B}-, and V3_{C}-FPs described above were also used. The “carrier” protein gp70 was also used as a control antigen in ELISA experiments; it was expressed in CHO cells and purified from culture supernatants as previously described (Krachmarov et al., 2001, supra). These proteins at concentrations of 0.4-1 μg/ml were coated onto wells of microplates (Immunolon 4, Dynatech, Chantilly, Va.) overnight at 4° C. Plates were washed with PBS/0.2% Tween-20 (PBST), serum samples were added with blocking buffer (5% FCS, 5% sheep serum, and 2.5% BSA in PBS) and incubated for 1 h at 37° C. After rinsing with PBST, anti-rabbit-IgG-HRP (Bio-Rad Laboratories, Hercules, Calif.) was added for 30 min. at 37° C. The samples were washed with PBS, developed with 100 μl TMB peroxidase substrate (KPL), the reaction stopped with 1M HCl, and absorbance was measured at 450 nm.

### Neutralization Assays

Neutralization of Primary Isolates

JC53-BL cells (also termed TZM-bl cells) were obtained from the NIH AIDS Research and Reference Reagent Program (catalog no. 8129). This is a genetically engineered HeLa cell clone that expresses CD4 and CCR5 and contains Tat-responsive reporter genes for firefly luciferase and Escherichia coli β-galactosidase under regulatory control of an HIV long terminal repeat. Cell lines were maintained in growth medium, consisting of Dulbecco's modified Eagle's medium (Gibco BRL Life Technologies), 10% heat-inactivated fetal bovine serum, 50 U/ml penicillin, 50 μg/ml streptomycin and 2 mM L-glutamine (BioWhittaker).

Neutralizing activity against primary isolates was measured as reductions in luc reporter gene expression after a single round of virus infection in JC53-BL cells as described previously (Li, M et al. (2005) J Virol. 79:10108-25). Briefly, 200 TCID₅₀ of virus was incubated with various dilutions of test samples for 1 h at 37° C. in a total volume of 150 μl growth medium in 96-well flat-bottom culture plates (Corning-Costar). For peptide inhibition studies, a 23-mer V3 peptide representing the V3 consensus sequence (TRPNNNTRKSIHIGPGRAFYTTG [SEQ ID NO:12]) was incubated for 30 min at a final concentration of 180 μg/ml with rabbit serum, and then 200 TCID₅₀ of virus in culture medium was incubated for 1 h at 37° C. (Bio-Synthesis, Inc., Lewisville, Tex.). Freshly trypsinized cells (10⁴) were added to each well and maintained in culture medium containing 1 μM indinavir sulfate. When necessary for efficient infection, DEAE-dextran was added to a final concentration of 25 μg/ml. The background control contained cells only, while the virus control contained cells plus virus. After 48 hr of incubation, 200 μl of medium was removed from each well and 50 μl of Bright Glo® reagent (Promega) was added. This was followed by a 2 min incubation at room temperature for cell lysis, transfer to 96-well black solid plates (Corning Costar), and measurement of luminescence using a Lumimark Plus microplate reader (BioRad). The percent reduction in relative luminescence units (RLU) was calculated relative to the RLU in the presence of preimmune serum. For all serum dilutions, the percent neutralization was calculated based on the RLU in the presence of immune sera from a given animal divided by the RLU in the presence of the same dilution of preimmune serum from the same animal. The 50% neutralizing titer was determined from the linear portion of the titration curve using the method of least squares.

Neutralization of Pseudoviruses (psVs)

Infectious pseudotyped viruses were generated by co-transfection of 293 cells with an env expression vector and with the complementing vector pNL4-3.Luc.R-E- (NIH AIDS RRRP, from Dr. Nathaniel Landau). Transfections were performed in tissue culture dishes using TransIT-LT1 Reagent (Mirus Bio Corporation, Madison, Wis.) according to the manufacturer's protocol. The env expression vectors for chimeric form of SF162 Env with various consensus V3 sequences were generated by introducing the modifications sequentially by QuikChange® site-directed mutagenesis (Stratagene, La Jolla, Calif.), as described by Krachmarov et al., 2006, supra.

Neutralization activity was determined per Krachmarov et al., 2001, supra, with a single-cycle infectivity assay using virions generated from the Env-defective luciferase-expressing pNL4-3.Luc.R⁻E⁻ genome (Connor, R I (1995) Virology 206:935-44) pseudotyped with a molecularly cloned HIV Env of interest. In brief, pseudotyped virions were incubated with serial dilutions of sera from immunized rabbits for 1.5 hour at 37° C., and then added to U87-T4-CCR5 target cells plated in 96-well plates in the presence of polybrene (10 μg/ml). After 24 hrs, cells were re-fed with RPMI medium containing 10% FBS and 10 μg/ml polybrene, followed by an additional 24-48 hr incubation. Luciferase activity was determined 48-72 hrs post-infection with a microplate luminometer (HARTA, Inc.) using assay reagents from Promega, Inc. Geometric mean titers for 90% neutralization (GMT₉₀) shown in Figures and Tables were determined by interpolation from neutralization curves and are averages of at least three independent assays.

### EXAMPLE II

### Design of Immunogens and Immunization Protocols

Two sets of rabbits were immunized. The protocol is summarized in Tables 2 and 3 (and described in more detail in Example I).

TABLE 2
Immunization groups for rabbit study

| Immunizing | | | |
|---|---|---|---|
| Regimen | Group | DNA prime at wks 0, 2, 4 | Protein boost at wks10 & 14 |
| —/B | I-1 | — | V3_{B}-FP |
| A_{R}/B | I-2 | gp120/Clade A (GPGR) | V3_{B}-FP |
| A_{R}/120_{R} | I-3 | gp120/Clade A (GPGR) | gp120_{JR-FL} |
| —/ABC | II-1 | — | V3_{A}-FP, V3_{B}-FP, V3_{C}-FP |
| A_{R}/ABC | II-2 | gp120/Clade A (GPGR) | V3_{A}-FP, V3_{B}-FP, V3_{C}-FP |
| C_{Q}/ABC | II-3 | gp120/Clade C (GPGQ) | V3_{A}-FP, V3_{B}-FP, V3_{C}-FP |
| A_{R} + C_{Q}/ | II-4 | gp120/Clade A (GPGR) and | V3_{A}-FP, V3_{B}-FP, V3_{C}-FP |
| ABC | | gp120/Clade C (GPGQ) | |
| A_{R}/B | II-5 | gp120/Clade A (GPGR) | V3_{B}-FP |

Note:
GPGR above is SEQ ID NO: 17;
GPGQ is SEQ ID NO: 17
*V3 sequences in priming and boosting constructs above are shown in Table 3, below. Variations in sequence from relevant consensus sequences are underlined and the variation at the tip of the loop, position 18 (R/Q), is bolded below (and bolded and underscored in Table 2)

TABLE 3

| | | |
|---|---|---|
| | | SEQ |
| | | ID |
| V3 Source | Sequence | NO: |

| | | |
|---|---|---|
| CA1 clade A1 env gp120 DNA prime (A_{R}): | CTRPNNNTRKGIHIGPGRAIYATGDIIGDIRQAHC | 13 |
| Clade C env gp120 DNA prime (C_{Q}): 92BR025.9 | CTRPNNNTRKSIRIGPGQAFYATGEIIGDIRQAHC | 14 |
| V3_{A}-FP from clade A strain 92UG037.08 | CTRPNNNTRKSVRIGPGQTFYATGDIIGDIRQAHC | 15 |
| V3_{B}-FP from clade B strain JR-CSF | CTRPSNNTRKSIHIGPGRAFYTTGEIIGDIRQAHC | 16 |
| V3_{C}-FP from clade C strain 93IN904 | CTRPNNNTRKSIRIGPGQTFYATGDIIGDIRQAHC | 6 |

The three rabbits in each group received three priming doses of codon-optimized gp120 DNA derived from env genes from primary isolates from clades A and/or C and two booster doses of gp120 or one or more V3-FPs. The rabbits were bled (1) before the commencement of the immunization protocol, (2) two weeks after the third DNA priming, and (3) two weeks after the second protein boost.

The gp120 genes from CA1 (an R5-tropic strain of CRF011_cpx) and from 92BR025.9 (an R5-tropic strain of clade C) were chosen for preparation of the DNA priming immunogen. The CA1 strain carries the gp120 of clade A and was selected on the basis of previous studies showing that its envelope was immunologically representative of a cluster of unrelated primary isolates from clades A, B, D, F and G (Nyambi P N et al., (2000) J Virol. 74:10670-80 and inventor's unpublished results). It is noteworthy that the CA1 V3 sequence contains the GPGR V3 motif ((SEQ ID NO:17) in Tables 2 and 3 which is present in only ˜4% of clade A envelopes (www.hiv.lanl.gov). The 92BR025.9 strain was chosen because it carries the V3 consensus sequence of clade C with the GPGQ motif (SEQ ID NO:18) at the tip of the V3 loop (Table 2 and 3).

For the protein boosts, gp120 from the JR-FL R5-tropic strain of clade B was used because it carries the V3 consensus sequence of clade B (with the GPGR motif) (SEQ ID NO:17). The V3_{JR-CSF}-FP (V3_{B}) was used as the boost because it is known to present the V3 epitope in its immunologically correct conformation (Kayman et al., supra), and because the V3 of the clade B JR-CSF strain differs from the clade B consensus sequence by only a single amino acid. The V3 part of V3_{A}-FP differs from the consensus sequence of clade A1 at two positions, and the V3_{C}-FP carries the consensus V3 sequence (see Tables 2-3) for that clade.

### EXAMPLE III

### Immunization with Monovalent Immunogens: Antibody Levels Measured by ELISA

In the first experiment, both the prime and boost constructs carried the GPGR V3 motif (SEQ ID NO:17). To compare the effect of priming and the boosting efficiency of gp120 vs. V3_{B}-FP, three groups of rabbits were used(see also Tables 2 & 3).

| | |
|---|---|
| Group I-1 (—/B): | no prime; immunized with V3_{B}-FP, |
| Group I-2 (A_{R}/B) | clade A DNA gp120 prime (carries GPGR V3 |
| | motif (A_{R}); boosted with V3_{B}-FP, and |
| Group I-3: (A_{R}/gp120_{R}) | clade A DNA gp120 prime followed by |
| | boosting with gp120 from the JR-FL clade B |
| | strain |

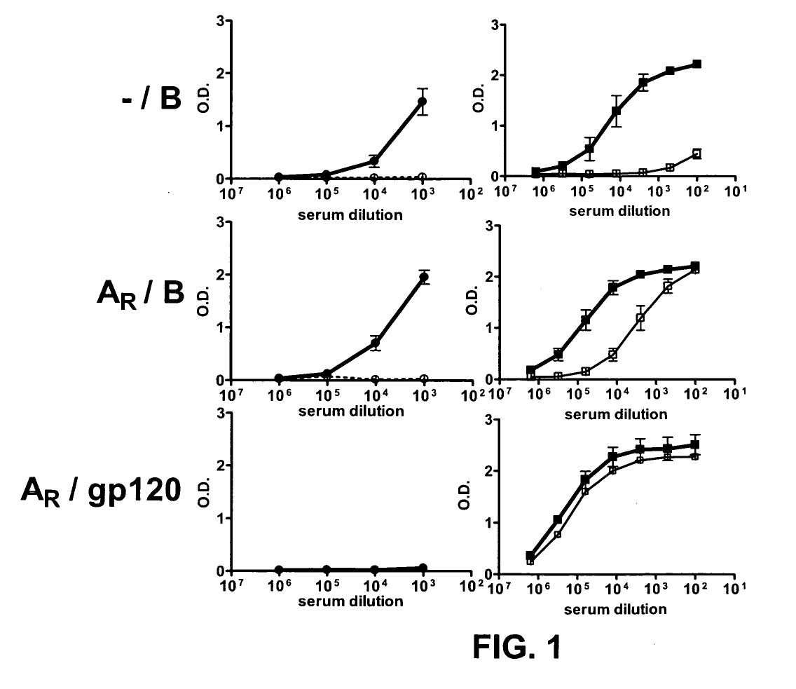

To determine the specificity of Abs induced by the various immunization regimens, the reactivities of the sera from immunized animals were measured against control MuLV gp70 (the protein into which the V3 sequences had been spliced to form the V3-FPs), against the YU-2 gp120 core, and against the YU-2 gp120 core carrying the V3 sequence (gp120+V3) (Wu, L et al. (1996) Nature 384: 179-83). The sera were derived from blood drawn prior to immunization (pre-bleeds) and/or from blood obtained two weeks after the second protein boost. The sera of animals that received V3-FP (Group I-1: -/B and Group I-2: A_{R}/B) made vigorous responses directed to the “carrier” gp70, whereas, as expected, the sera of rabbits not receiving (Group I-3: A_{R}/gp120_{R}), and the pre-bleed sera from all three groups, had no detectable anti-gp70 Abs (FIG. 1, left column).

To determine the levels of anti-V3 responses, serum reactivity was tested against gp120 core and gp120 core+V3 (FIG. 1, right column). As expected, sera drawn two weeks after the second boost from rabbits of Group I-1:-/B, which received only V3-FP, reacted only with gp120 core+V3 and displayed essentially no binding activity against gp120 core. Sera from rabbits of Group I-2: A_{R}/B displayed titers against gp120 core+V3 that were significantly greater than titers against gp120 core (1:69,398 vs. 1:2,909, respectively).

This pattern demonstrated that boosting with a V3-FP was able to focus the Ab response on the V3 epitope. In contrast, sera of rabbits primed with gp120 DNA and boosted with gp120 (Group I-3: A_{R}/gp120_{R}) displayed similar Ab reactivities against the gp120 core and gp120 core+V3 (1:147,365 and 1:217,126, respectively). Thus, when gp120 protein was used as the boosting immunogen, the V3 region is not an immunodominant epitope.

### EXAMPLE IV

### Immunization with GPRG-Based Immunogens: Neutralization of Primary Isolates

As described above, a multi-tier approach has been recommended for assessing the neutralizing Ab responses generated by candidate HIV vaccines (Mascola, J R et al., supra. These recommendations suggested that, initially, immune sera should be tested against “Tier 1 viruses” which consist of “homologous virus strains represented in the vaccine and a small number of heterologous viruses that are known to be highly sensitive to Ab-mediated neutralization”. Subsequently, testing should be undertaken against “Tier 2 viruses” (heterologous viruses that match the genetic subtype of the vaccine) and “Tier 3 viruses” (a multi-clade panel of Tier 2 viruses). Although no Tier 1 panels have been specified, it is generally acknowledged that Tier 1 viruses are sensitive to Abs that are specific for V3 and/or CD4i Abs; SF162 and MN are the only two primary isolates currently acknowledged and cited as Tier 1 viruses (Law, M et al. (2007) J Virol. 81:4272-85).

In the absence of a Tier 1 panel, primary isolates were selected based on previous studies showing the ability of anti-V3 mAbs at 25 μg/ml to achieve 50% neutralization of these viruses (Gorny, M K et al. 2006, J Virol 80:6865-72). The viruses selected include CA1 (CRF011_cpx, one of the strains used in the vaccine prime), DJ263 (CRF02_AG), BX08 (clade B), and NYU129/5 (CRF02_AG). These viruses are more resistant to anti-V3 mAbs than SF162, and so should more accurately be identified “Tier 1+” viruses. However, for the sake of brevity, they are will designated here as Tier 1 viruses. In addition, primary isolates previously shown to be still more resistant to neutralization were tested (Gorny et al., 2004. supra; Gorny et al., 2006, supra) that were heterologous to the strains on which the immunogens were based and might therefore be categorized as Tier 2 and 3 viruses, respectively. These included JR-FL (clade B), 98CN006 (clade C), 93MW965 (clade C), and 93MW960 (clade C).

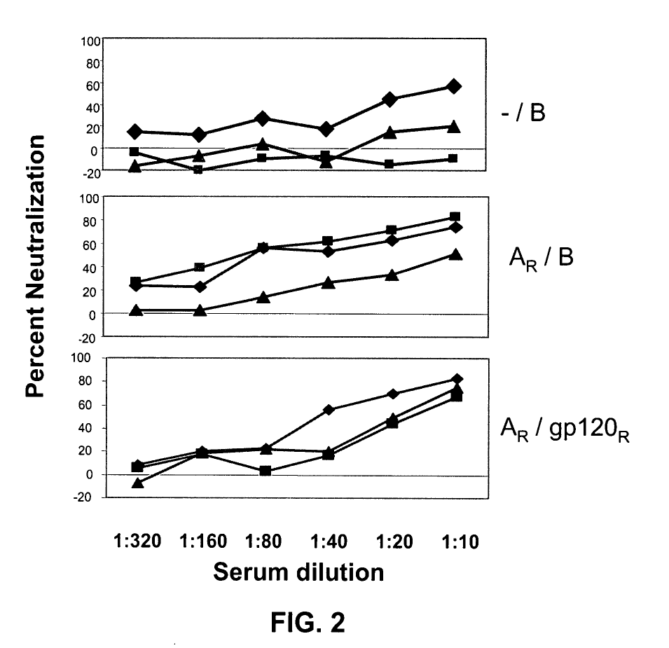

Neutralizing activity in the sera of the rabbits in the first set of experiments was tested against primary isolate DJ263, a virus from CRF02_AG whose V3 loop carries the GPGQ motif (SEQ ID NO:18). Results of the titration of the neutralizing activity are shown in FIG. 2 for sera drawn two weeks after the second protein boost. Sera from Group I-1: -/B displayed little or no neutralizing activity; the geometric mean titer for 50% neutralization (GMT₅₀) in this group derived from two experiments was 1:13 (Table 4).

TABLE 4
Neutralizing Activity in Immune Rabbit Sera

| | | DJ263 (CRF02_AG) | | BX08 (Clade B) | | CAI (CRF011_cpx) | |
|---|---|---|---|---|---|---|---|
| | Immunizing | | % Neutralization | | % Neutralization | | % Neutralization |
| Group | Regimen^{‡} | GMT₅₀* | due to V3 Abs | GMT₅₀* | due to V3 Abs | GMT₅₀* | due to V3 Abs |
| I-1 | —/B | 1:11 | 90%^{¥} | 1:50 | 79% | <1:10 | — |
| I-2 | A_{R}/B | 1:46 | 88% | 1:186 | 69% | 1:29 | 68% |
| I-3 | A_{R}/gp120_{R} | 1:23 | 33% | 1:320 | 38% | 1:66 | 49% |

^{‡}Defined in Table 2 and 3.
*Mean of values from all rabbit sera in each group tested in two separate experiments.
^{¥}Based on neutralization of DJ263 by sera from this group that contained neutralizing antibodies

In contrast, the GMT₅₀ calculated from two experiments for all three rabbits in Group I-2: A_{R}/B was 1:81. All rabbits in Group I-3: A_{R}/gp120_{R}, also mounted a significant NAb response, with a GMT₅₀ of 1:36 based on results from two experiments. It is particularly noteworthy that all rabbits in this experiment were immunized with constructs derived from Env carrying V3 sequences with GPGR (SEQ ID NO:17), but these rabbits' antisera were able to neutralize a primary isolate carrying the GPGQ motif (SEQ ID NO:18).

A dose-response relationship was demonstrated in the neutralization assay (FIG. 2), and, when the immunization regimen focused the immune response on the clade B V3 loop by using the V3-FP_{B} boost (Group I-2: A_{R}/B), the neutralizing GMT₅₀ reached levels comparable to those achieved by administration of the entire gp120 molecule (Group I-2: A_{R}/gp120_{R} or that had otherwise required the use of polyvalent cocktail of full-length gp120 molecule delivered as both DNA and protein (Wang et al., 2006, supra).

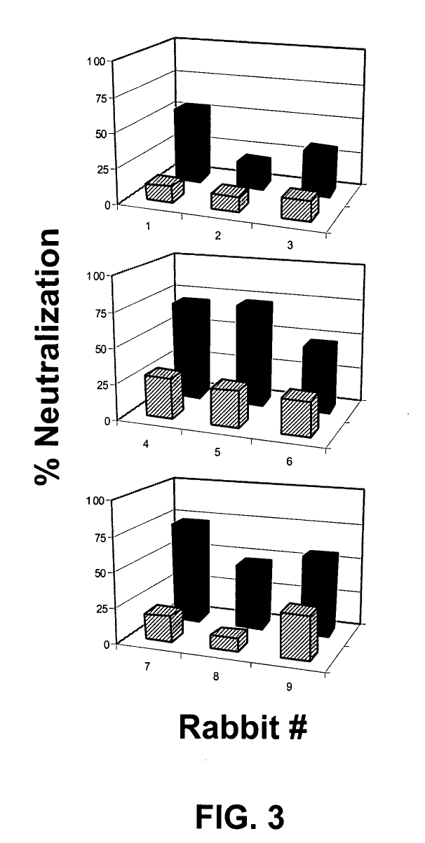

Moreover, the results reported show substantially stronger NAb responses than those previously achieved for cross-clade neutralizing activity in immunized animals against primary isolates using other immunization approaches (Chakrabarti et al., supra; Lian et al., supra) The efficacy of the boost is shown in FIG. 3: there was minimal neutralizing activity against DJ263 in sera obtained two weeks after the third DNA prime (before any boosting) vs. the activity of sera obtained two weeks after the second protein boost. These results confirmed earlier findings (Wang et al., 2006, supra) that DNA immunogen priming alone induces a barely detectable level of NAbs, whereas the protein boost is primarily responsible for the induction of clearly positive NAb responses. For this reason, all subsequent results are shown for sera drawn two weeks after the second boost.

As noted above (see FIGS. 2 and 3), there seemed to be little quantitative difference in the neutralizing activity in sera from rabbits in Groups I-2: A_{R}/B and I-3: A_{R}/gp120_{R}, however the ELISA results (shown in FIG. 1) demonstrated a qualitative difference in the specificity of the Abs, with the V3_{B}-FP-primed group (I-2: A_{R}/B) and the gp120-primed group (I-3: A_{R}/gp120_{R}) having different profiles of Abs reactivities.

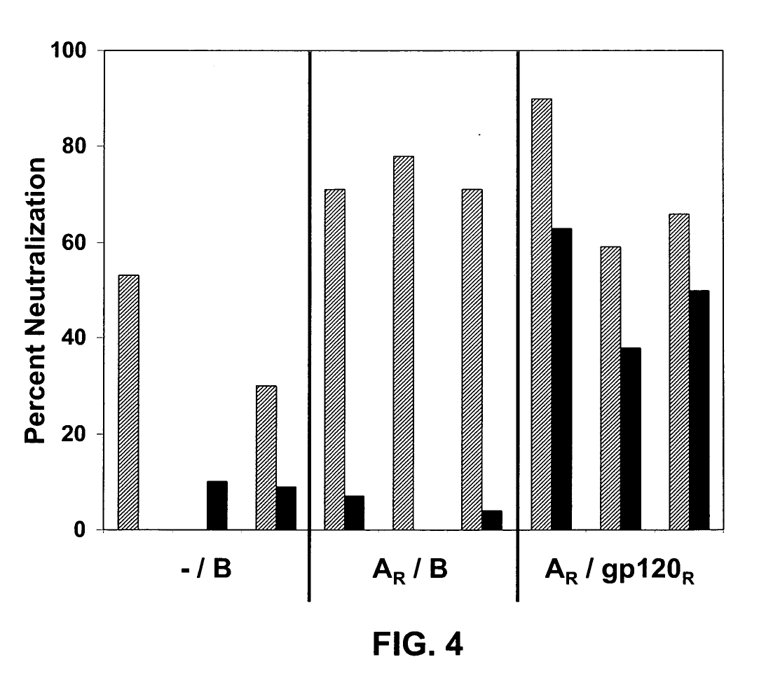

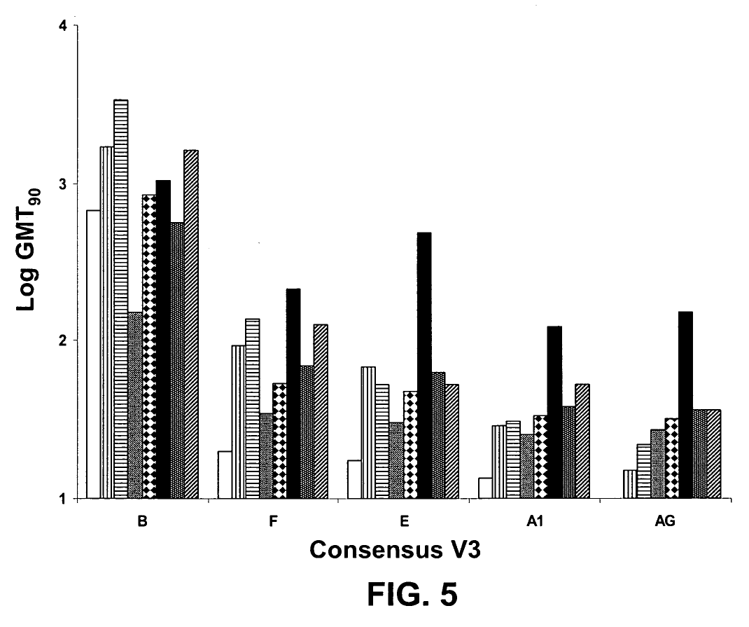

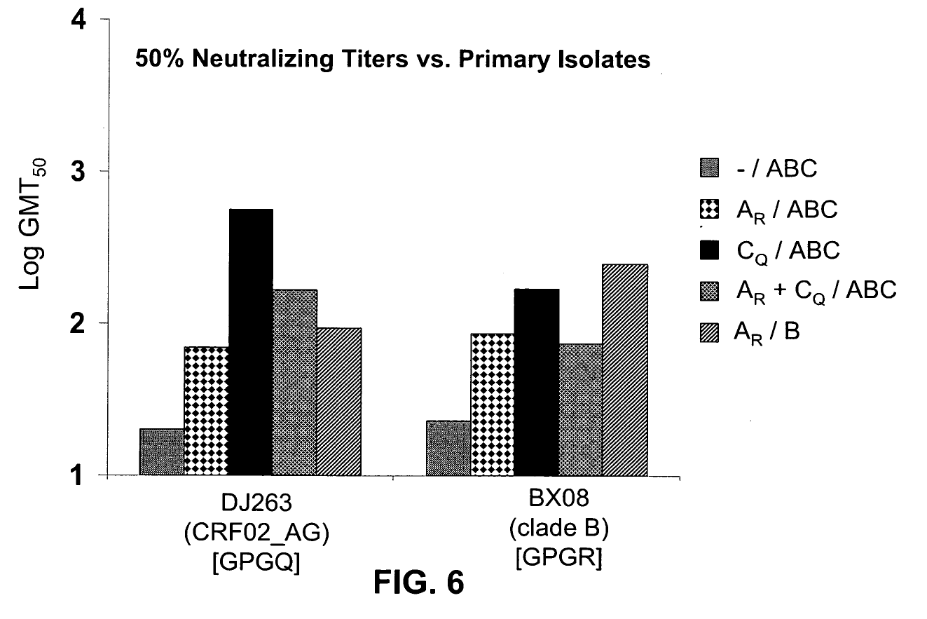

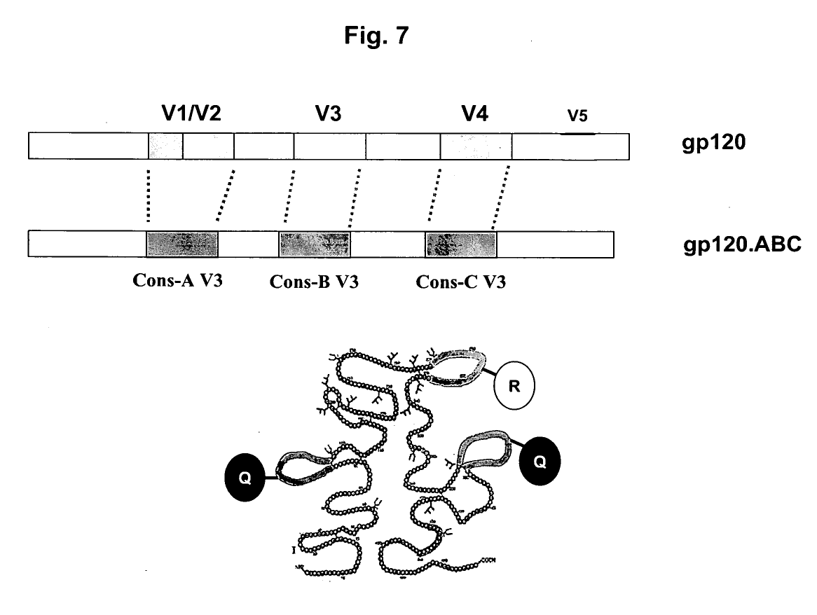

To determine if there was also a qualitative difference in the specificity of the NAbs in the sera from the different groups, sera were tested at a 1:20 dilution with or without pre-incubation with a V3 peptide. FIG. 4 shows a representative experiment measuring neutralizing activity against DJ263. The results confirm that the sera from Groups I-2 and I-3 are quantitatively similar, with sera diluted 1:20 giving ˜60-90% neutralization. However, the sera from the rabbits in these groups show a substantial qualitative difference. Thus, the majority of the neutralizing activity in sera from Group I-2: A_{R}/B was removed by pre-incubation with the V3 peptide. In contrast, pre-incubation with V3 peptide only partially reduced the neutralizing activity of sera from Group I-3: A_{R}/gp120_{R}. The sera from two of the animals in Group I-1: -/B showed weak neutralizing activity, and most or all of the activity was due to anti-V3 Abs, as expected. Pre-incubation of immune sera with 180 μg/ml of a scrambled peptide with the same amino acids composition as the V3_{B} consensus 23-mer peptide did not result in any significant absorption (and therefor reduction or loss) of neutralizing activity.

Similar peptide inhibition experiments were performed to identify the proportion of Abs that neutralize Tier 1 viruses BX08 (clade B primary isolate) and CA1 (clade A1). The results (see Table 4 and FIG. 5) again show that the use of the V3-FP results in NAbs that are preferentially V3-specific. Thus, 69% and 68% of the BX08 and CA1 neutralizing activity, respectively, in the sera of the Group I-2: A_{R}/B rabbits were blocked by V3 peptide, where as 38% and 49% of the comparable neutralizing activity was blocked in the sera of the Group I-3: A_{R}/gp120_{R} rabbits (FIG. 5). Thus, while boosting with V3-FP induces a response that is quantitatively similar to that achieved with whole gp120, using a boosting immunogen with a single neutralizing epitope is able to focus the immune response such that NAbs to that epitope are preferentially produced. Immune sera from the rabbits receiving the GPGR-based vaccine regimen were also tested against Tier 2 (JR-FL; clade B) and Tier 3 (98CN006, 93MW965, 93MW960; all clade C)) viruses. 50% neutralization was not detected at final serum dilutions of 1:20.

### EXAMPLE V

### Immunization with GPGR-Based Immunogens: Neutralization of V3 Chimeric Pseudoviruses

For many primary isolates, the V3 loop is partially or fully masked by the V1/V2 loop. To assess the cross-neutralizing activity of anti-V3 Abs, viruses with unmasked V3 loops can be used; one such virus is the clade B strain SF162 (Krachmarov et al., 2005, supra). To determine the extent of cross-clade neutralizing anti-V3 activity in the immune rabbit sera, V3 chimeric pseudoviruses were constructed in which the V3 loop of SF162 was replaced with the consensus V3 sequences from clades A1, B, C, F, H, CRF01_AE, and CRF02_AG (for example, the results in FIG. 5). The GMT₉₀ of the pre-bleed sera tested against these seven V3 chimeric pseudoviruses was <1:10.

In the immune sera of rabbits from this experiment, the GMT90 were consistently highest against the pseudovirus carrying the clade B V3 consensus sequence, reflecting the preference for the GPGR motif (SEQ ID NO:17) at the tip of the loop which is homologous to that in both the priming and boosting immunogens used (FIG. 5). The GMT₉₀ for Group I-1: -/B, Group I-2: A_{R}/B, and Group I-3: A_{R}/gp120_{R} against the pseudoviruses carrying the consensus V3 sequence of clade B were 1:689, 1:1717, and 1:3308, respectively. While the GMT₉₀ were <1:10 for neutralizing activity in the sera from animals in each group against pseudoviruses carrying the consensus V3 sequences of clades C or H (not shown), neutralizing activity against pseudoviruses carrying the consensus V3 loops of clades A1, F, AE, or CRF02-AG were detected at levels of 1:22 to 1:136 in the sera of animals that had been primed and boosted with either V3-FP or gp120 (Group I-2: A_{R}/B and Group I-3: A_{R}/gp120_{R}; FIG. 5). These results demonstrate the induction of NAbs that can recognize viral envelopes bearing the V3 loops of diverse clades.

### EXAMPLE VI

### Immunization with Multivalent Immunogens: Anti-V3 Binding Activity

A second set of rabbits was immunized using multivalent priming and/or boosting (Table 2). The multivalent approach was based on previous work showing that broader immune responses could be elicited with immunogens derived from diverse HIV clades (Wang et al., 2006, supra); Lian et al., supra; Chakrabarti et al., supra). The sera of animals receiving the multivalent vaccine regimen obtained two weeks after the second protein boost, were titrated for their binding activity against V3_{A}-, V3_{B}- and V3_{C}-FPs. The results shown in Table 5 demonstrate that the strongest response to the three V3-FPs was mounted by rabbits in Group II-3: C_{Q}/ABC.

TABLE 5
Reciprocal half-maximal binding titers to V3A-V3B- and V3C-FPs of
immune sera obtained two weeks after second boost

| Group* | V3_{A}-FP | V3_{B}-FP | V3_{C}-FP |
|---|---|---|---|
| —/ABC | 1,765^{¶} | 6,556 | 1,418 |
| AR/ABC | 1,869 | 16,469 | 1,869 |
| CQ/ABC | 5,580 | 27,332 | 5,268 |
| AR + CQ/ABC | 3,243 | 20,106 | 3,020 |
| AR/B | 1,651 | 27,227 | 1,354 |

*Groups as defined in Table 2 and 3.
^{¶}Reciprocals of the geometric means of the titers from the sera of each of the three rabbits in each group.

### EXAMPLE VII

### Immunization with Multivalent Immunogens: Neutralization of Primary Isolates

To determine if multivalent immunogens would help to broaden the immune response when, simultaneously, the immune response was focused on a single neutralizing epitope, animals received either no DNA priming (Group II-1: -/ABC), a gp120 DNA prime based on the clade A_{R} env (Group II-2: A_{R}/ABC), a gp120 DNA prime based on the clade C_{Q} env (Group II-3: C_{Q}/ABC), or a combined clade A_{R} and C_{Q} gp120 DNA prime (Group II-4: A_{R}+C_{Q}/ABC). All animals in these groups received boosts of cocktail of V3_{A}- V3_{B}- and V3_{C}-FPs. Group II-5: A_{R}/B serves as “benchmark,” recapitulating Group I-2: A_{R}/B in the previous set of rabbits.

The immune sera from the rabbits in this experiment were again tested first for their ability to neutralize Tier 1 primary isolates which, in this case, included CA1 (CRF02_AG) and 92BR025 (clade C), each used as the basis of the gp120 DNA prime, and BX08 (clade B), DJ263 (CRF02_AG), and NYU129/5 (CRF02_AG). While no significant neutralizing activity was detected in the sera of any of the rabbits when tested at a dilution of 1:20 against NYU129/5 or 92BR025 (not shown), the neutralizing activity demonstrated against primary isolates DJ263 and BX08 and CA1 is shown in FIG. 6. The rabbits that received the C_{Q}/ABC regimen (Group II-3) displayed the strongest response against DJ263 (GMT₅₀ of 1:559), the virus that carries the GPGQ V3 motif also found in the C_{Q} gp120 DNA prime and in the V3_{A}- and V3_{C}-FPs used to boost. In contrast, the sera from Group II-5: A_{R}/B showed the strongest reactivity against primary isolates BX08 and CA1 carrying the GPGR V3 motif. Thus, sera from Group II-5: A_{R}/B displayed a GMT₅₀ vs. clade B virus BX08 of 1:246, and a GMT₅₀ vs. CA1 (the CRF011_AG virus from which the A_{R} boost was constructed and which contains a GPGR V3 motif) of 1:111 (FIG. 6).

The sera of the animals immunized with the multivalent vaccine regimen were also tested against Tier 2 viruses including JR-FL (clade B), 98CN006 (clade C), 93MW960 (clade C), and 93MW965. Fifty percent neutralization was not detected against any of these Tier 2 primary isolates when tested at a final serum dilution of 1:20.

### EXAMPLE VIII

### Immunization with Multivalent Immunogens: Neutralization of Pseudoviruses

Neutralization experiments were next performed using the panel of pseudoviruses made with the SF162 Env or chimeric forms of this Env carrying the consensus V3 sequences from clades A1, AG, B, C, F, AE, C and H. The neutralization data with psVs (FIG. 5) support data from assays against the Tier 1 and 2 viruses showing that Group II-3: C_{Q}/ABC mount the broadest response (FIG. 6). The response to the psV carrying the clade B V3 consensus sequence was strong (NT₉₀>1:100) in all animals that received a prime and boost, but the response to the psVs carrying the GPGQ V3 motif in consensus V3 sequences of clades F, E, A1 and AG were consistently highest when clade C gp120 DNA was used to prime and V3_{A}-, V3_{B}- and V3_{C}-FPs were used to boost (Group II-3: C_{Q}/ABC, FIG. 5) It is only in this latter group that rabbit immune sera achieve a GMT₉₀>1:100 for the clade B, F. E, A1 and AG V3 chimeric psVs. GMT₉₀ levels≧1:10 were not achieved by sera from any of the rabbit groups against psVs carrying the clade C or H consensus V3 sequence. However, GMT₅₀ levels of neutralizing Abs against these latter psVs were achieved by all groups of animals receiving both DNA prime and protein boosts, with titers ranging from 1:15 to 1:85. Group II-3: C_{Q}/ABC again achieved the highest levels of Abs, with GMT₅₀ vs. clade C and H chimeric psVs of 1:85 and 1:53, respectively (results not shown).

The sera from these animals receiving the multiclade immunofocusing regimen were also assayed against the Tier 2 standard clade B panel of psVs (Li et al., supra). None of the rabbit sera achieved 50% neutralization at titers of 1:10

### EXAMPLE VIII

### Priming with Multiclade Immunogens

An additional primer was designed based on replacing in the DNA encoding gp120, the V1/V2 and V4 regions/loops with additional V3 peptides. In fact, the preferred first design involved replacing (i) the V1/V2 loop in gp120 with a consensus V3 sequence of clade A, (ii) the native V3 with a consensus V3 sequence of clade B, and (iii) the V4 loop with the consensus V3 sequence of clade C. This construct is schematically illustrated in FIG. 7. The upper portion is a linear schema of a “native” gp120 and this new gp120 designated gp120.ABC (or gp120_{ABC}). The lower portion of the Figure shows the secondary structure of gp120 indicating the replaced loops, pointing out additionally the “tips” of the loops have the sequence GPGR (SEQ ID NO:17) in the clade B consensus sequence, but are GPGR (SEQ ID NO:18) in the clade A and clade C V3 consensus sequences.

The above DNA construct was used to prime rabbits using the methods described above. This is shown in the upper part of Table 6, which shows 50% neutralizing titers (or ND₅₀) of primary isolates from clade B, A, and C with sera from mice primed with gp120_{ABC} DNA and boosted with a mixture of V3_{A}-FP, V3_{B}-FP and V3_{C}-FP. (The lower part of the Table shows results of priming with a p120 that has a single (GPGR-containing) V3 loop.

These results show that a V3_{ABC} priming immunogen stimulates potent cross-clade Nabs.

TABLE 6
50% Neutralization Titers vs. Tier 1 and Tier 2 Primary Isolates

| | | Primary Isolates with Envelope from: | | | | | | |
|---|---|---|---|---|---|---|---|---|
| DNA prime/ | | clade B | | clade A | | clade C | | |
| Protein Boost | rabbit | BZ167 | BX08 | CA1 | DJ263 | 92BR025 | 93MW965 | 98CN006 |
| gp120_{ABC}/ | 31 | 317 | 218 | <10 | 252 | <10 | <10 | <20 |
| V3_{A} + V3_{B} + V3_{C} | 32 | >540 | 117 | 23 | 459 | 11 | <10 | <20 |
| | 33 | <20 | 62 | <10 | 447 | <10 | <10 | <20 |
| | 34 | >540 | 125 | <10 | 288 | <10 | <10 | <20 |
| | 35 | 64 | 55 | <10 | 228 | <10 | <10 | <20 |
| gp120(GPGR)/ | 36 | <20 | 28 | 14 | 379 | 229 | >160 | 49 |
| V3_{A} + V3_{B} + V3_{C} | 37 | >540 | 234 | 46 | 404 | 72 | 15 | 21 |
| | 38 | 90 | 219 | 49 | 40 | 19 | 15 | <20 |
| | 39 | 45 | 479 | 48 | 191 | 145 | 43 | 59 |
| | 40 | >540 | 214 | 74 | 442 | <10 | <10 | <20 |

Numbers represent 50% Neutralization Titers

TABLE 7
Increasing Breadth and/or Potency of Antibody Response

| | | | Median ND₅₀ vs. V3 Chimeric Pseudoviruses Carrying the | | | | | | |
|---|---|---|---|---|---|---|---|---|---|
| | gp120 DNA | Protein | Consensus V3 Loops from the following Clade: | | | | | | |
| Protocol | prime | boost | B | F | A1 | E | AG | C | H |
| NYU-1 | Control Vector | V3_{B}-FP | 6,767 | 123 | 106 | 138 | 32 | <1:10 | <1:10 |
| | A (GPGR)* | V3_{B}-FP | 14,540 | 1,017 | 167 | 1,191 | 152 | <1:10 | 18 |
| | A (GPGR) | gp120 | 14,683 | 1,617 | 271 | 318 | 134 | 24 | 48 |
| NYU-2 | Control Vector | V3_{A,B,C}-FP | 415 | 40 | 69 | <1:10 | 48 | <1:10 | <1:10 |
| | A (GPGR) | V3_{A,B,C}-FP | 10,583 | 400 | 287 | 230 | 178 | 19 | <1:10 |
| | C (GPGQ)* | V3_{A,B,C}-FP | 12,200 | 1,935 | 1,603 | 2,125 | 1,897 | 85 | 86 |
| | A (GPGR) + C | V3_{A,B,C-}FP | 5,133 | 682 | 338 | 873 | 252 | 20 | 30 |
| | (GPGQ) | | | | | | | | |
| NYU-3 | “gp120_{ABC}” | V3_{A,B,C}-FP | 4,350 | 670 | 270 | ND | 277 | 171 | <1:10 |
| | A (GPGR) | V3_{A,B,C}-FP | 5,263 | 1,016 | 140 | ND | 148 | 18 | <1:10 |

*GPGR is SEQ ID NO: 17; GPGQ is SEQ ID NO: 18
V3_{A,B,C}-FP refers to a mixture of three different fusion proteins comprising clade A, B and C V3 loops.

TABLE 8
Neutralization titers (ND₅₀) of Rabbit Sera from Animals primed with Clade C gp120 DNA
and boosted indicated Tested Against HIV Isolates of Different Clades

| | | | | | | | | | Test on Clade AG virus | | | | | | | |
|---|---|---|---|---|---|---|---|---|---|---|---|---|---|---|---|---|
| | | Test on Clade B virus | | | | Test on Clade A1 | | | | NYU- | | Test on Clade Cv virus | | | | |
| | | BX | BZ | | NYU- | virus | | | | 6525 | NY129 | 97ZA | 98CN | 92BR | 93MW | 93MW |
| Boost* | Rabb # | 08 | 167 | CA5 | 3738 | VI191 | VI313 | CA1 | DJ263 | (2) | (5) | 009 | 006 | 025 | 965 | 960 |
| A | 41 | 24 | 45 | | | | | | 98 | | +/− | | | | | |
| | 42 | 50 | 502 | 14 | | +/− | 12 | | 382 | | | | | | | |
| | 43 | 92 | 401 | 26 | | 10//13 | 15 | +/− | 652 | | | | | | +/− | 10 |
| | 44 | | 97 | | | | 16 | | 427 | | +/− | | +/− | | | |
| | 45 | 40 | 53 | | | | | | 237 | | | | | | | |
| B | 46 | 31 | 24 | | | +/− | 41 | | 186 | | +/− | | | | | |
| | 47 | 110 | 777 | 10 | | | 16 | +/− | 582 | | | | | | | |
| | 48 | 225 | 637 | 11 | | | 185 | +/− | >540 | | | | | | | |
| | 49 | 41 | 12 | 42 | 23 | 23/42 | 54 | | 328 | | 17/29 | | 30/14 | 12 | 18/21 | 28 |
| | 50 | 73 | 69 | 28 | 23 | 26/30 | 32 | | 389 | >20 | 19/50 | | 26/33 | 21 | 30/26 | 25 |
| C | 51 | 45 | 690 | 11 | | | 18 | +/− | 305 | | | | | | | |
| | 52 | 16 | 335 | 15 | | +/− | | | 292 | | | | | | | |
| | 53 | 20 | 103 | 10 | | +/− | | | 122 | | +/− | | | | | 12 |
| | 54 | 16 | 961 | 24 | 15 | 16/24 | 23 | | 470 | | 11/17 | | 19/12 | 10 | 16/17 | 24 |
| | 55 | 26 | 153 | 28 | 27 | 38/33 | 30 | | 423 | >20 | 30/22 | 10 | 35/35 | 24 | 31/61 | 36 |
| A + C | 56 | 37 | 74 | | | | | | 195 | | +/− | | | | | |
| | 57 | 44 | 624 | 15 | | +/− | | | 297 | | +/− | | 10/10 | | | 11 |
| | 58 | 63 | 243 | 16 | 13 | 16/16 | 37 | | 617 | | 13/13 | | 17/11 | | 10/20 | 18 |
| | 59 | 44 | 221 | 12 | | 10/15 | 16 | | 174 | | 11/13 | | +/− | 10 | 10/11 | 14 |
| | 60 | 103 | 176 | | | | | | 290 | | | | | | | |
| A + B + C | 61 | 41 | 118 | | | | | | 155 | | | | | | | |
| | 62 | 75 | 60 | 12 | | +/− | 23 | | 374 | | 10/11 | | +/− | | 10/10 | 10 |

β* rabbits were primed three times with clade C gp120 DNA *gp120(C_{Q})” and boosted twice with the indicated fusion protein (FP) comprising the V3 consensus sequence of clade A, B or C, or mixtures of A + C or A + B + C. The values are reciprocal serum dilutions which gave 50% neutralization of each virus.
Completed viruses are indicated in bold; blanks indicate that 50% neutralization was not reached even at the highest serum concentration tested. It does not mean that those sera were not tested. All sera were tested in all combinations above.
Two values separated by a “/” are from two separate experiments.

TABLE 9
Neutralizing Titers (ND₅₀) sera from rabbits boosted with various V3 protein combinations
against Pseudotyped SF162 HIV-1 virions

| | | | V3 Sequence from | | | | | | | |
|---|---|---|---|---|---|---|---|---|---|---|
| | | | Clade B | | V3 Sequence from other clades | | | | | |
| | | | | SF162, | | | CRF02_AG | | | |
| | | | | consensus | | | Clade | CRF01_AE | | |
| DNA | Protein | | SF162 | V3 | Clade A1 | Clade F | “A/G” | Clade “AE” | Clade C | Clade H |
| Prime* | Boost | Rabbit # | p782 | (p1531) | p1522 | p1534 | p1441 | p1520 | p1515 | p1530 |
| C1 opt | A | 41 | 250 | 3,300 | 750 | 610 | 400 | 300 | <10 | <10 |
| | | 42 | 580 | 8,000 | 5,700 | 2,450 | 1,650 | 160 | 170 | <10 |
| | | 43 | 1,100 | 25,500 | 4,000 | 3,450 | 1,050 | 28 | 69 | <10 |
| | | 44 | 230 | 8,800 | 1,650 | 3,150 | 1,200 | 950 | 12 | 39 |
| | | 45 | 135 | 1,700 | 1,950 | 760 | 1,250 | 95 | 72 | 12 |
| C1.opt | B | 46 | 260 | 10,500 | 1,200 | 690 | 410 | 300 | 42 | 28 |
| | | 47 | 560 | 40,000 | 1,900 | 1,650 | 700 | 3,100 | 180 | 58 |
| | | 48 | 2,150 | 62,000 | 5,950 | 6,240 | 2,850 | >6250 | 285 | 430 |
| | | 49 | 540 | 36,000 | 3,800 | 2,100 | 2,150 | >6250 | 145 | 23 |
| | | 50 | 620 | 47,000 | >6250 | 2,950 | 2,800 | >6250 | 210 | 25 |
| C1.opt | C | 51 | 215 | 8,200 | 4,400 | 790 | 1,900 | 3,800 | 20 | 36 |
| | | 52 | 105 | 4,300 | 4,100 | 630 | 900 | 230 | 18 | 20 |
| | | 53 | 73 | 2,650 | 620 | 330 | 1,100 | 17 | 20 | <10 |
| | | 54 | 70 | 2,200 | 1,750 | 460 | 2,950 | 280 | 105 | <10 |
| | | 55 | 88 | 2,950 | 2,300 | 1,600 | 1,600 | 2,700 | 105 | <10 |
| C1.opt | A + C | 56 | 135 | 5,000 | 3,400 | 1,000 | 900 | 550 | 75 | <10 |
| | | 57 | 500 | 11,000 | 530 | 650 | 1,450 | 680 | 27 | 16 |
| | | 58 | 455 | 13,500 | 600 | 830 | 1,100 | 3,400 | <10 | 24 |
| | | 59 | 425 | 5,850 | 230 | 350 | 740 | 920 | <10 | 27 |
| | | 60 | 575 | 11,300 | 600 | 760 | 920 | 190 | 16 | 100 |
| C1.opt | A + B + C | 61 | 240 | 9,100 | 440 | 830 | 1,400 | 1,200 | 500 | 25 |
| | | 62 | 145 | 40,000 | 570 | 830 | 780 | 300 | 560 | 14 |
| | | Mean | 430 | 16,311 | 2,211 | 1,505 | 1,373 | 1,011 | 138 | 58 |

The priming and boosting was as described for Table 8

Table 7 compares the results of several different approaches and testing on V3 chimeric pseudoviruses carrying consensus V3 loops from 7 different clades.

| | |
|---|---|
| (NYU-1): | priming with a single V3 of clade A and boosting with either a clade B V3-FP or with |
| | a complete gp120 protein. Cross-clade Nabs were induced in this manner (see also |
| | above). |
| (NYU-2): | priming with gp120 of either clade A or clade C or a combination of the two and |
| | boosting with a cocktail of three V3 proteins (of clade A, B and C). Again, potent |
| | cross-clade neutralization was induced. |
| (NYU-3): | related to the study shown in Table 6, a multiclade immunogen (gp120_{ABC}) was used |
| | to prime rabbits (in comparison with priming gp120 with V3 of a single clade), and |
| | boost with a cocktail of three V3 proteins (clade A, B and C). This differs from the |
| | study in Table 6 in that here, neutralization was tested on pseudoviruses, not actual |
| | human HIV isolates. Again, potent cross clade NAbs were evident, and the multiclade |
| | priming immunogen induced stronger responses against some of the non-A, non-B |
| | and non-C clades. |

### EXAMPLE IX

### Responses After Priming with a GPGQ-Containing gp120 and Boosting with Various Combinations

A study referred to as NYU-4 examined responses from animals primed with a gp120 of clade C wherein the gp120 had the GPGQ sequence (SEQ ID NO:18) in the V3 loop. This sequence is the most frequent in the HIV-1 viruses responsible for natural human infections. The immunization protocol is indicated in the table below:

| Group | gp120 DNA Prime × 3 | Protein Boost × 2 |
|---|---|---|
| Group IV-1 | gp120(C_{Q}) | V3_{A}-FP |
| Group IV-2 | | V3_{B}-FP |
| Group IV-3 | | V3_{C}-FP |
| Group IV-4 | | V3_{A}-FP + V3_{C}-FP |
| Group IV-5 | | V3_{A}-FP + V3_{B}-FP + V3_{C}-FP |

Sera from rabbits immunized in this way were tested first against primary HIV-1 isolates of different clades (B, A1, A/G and C) and found to show relative high NAbs responses (considering the targets are real viral isolates) against multiple clades in addition to that of the priming and boosting immunogens.

Finally, sera from the same immunizations discussed above were tested against HIV-1 viral pseudovirions in which V3 of various clades (B, A2, A/G, C) had been engineered into the clade B SF162 strain. Results shown in Table 9, above, indicate that measurable cross-clade NAb responses were induced by the various boosting regiments. High titers of cross clade neutralization were particularly evident with clades F. A1. AE and AG.

The inventor concluded from the foregoing that the immunization regiments of the present invention induce Abs in rabbits that neutralize HIV viruses from more than 1 clade. A neutralizing epitope, presented on a non-HIV scaffold, induces neutralizing Abs. The immune response can be focused on a single neutralizing epitope and NAbs can be induced with a titer of ≧1:20 against at least 6 “Tier 1” viruses representing at least 2 clades. Importantly, this invention permits crossing the “Q/R barrier” in that immunization as described herein against a HIV clade characterized by the GPGR sequence (SEQ ID NO:17) in its V3 loop can result in NAbs that act on viruses having the GPGQ sequence (SEQ ID NO:18) in their V3 loops and vice versa, thus opening the door to a broader array of effective vaccines useful against the majority of natural HIV infections (in which V3 has GPGQ).

### Further Discussion of Examples

The results described here demonstrate the feasibility of focusing the immune response on a single protein domain that elicits NAbs. The results prove the principle that the immune response can be focused on selected regions of the HIV envelope, that the majority of NAbs elicited can, indeed, be targeted to the selected epitope, and that a broad response can be elicited with this technique. While the vaccine constructs used in the foregoing Examples were designed to focus the immune response on only a single HIV Env epitope, the V3 loop, the incorporation of selected additional neutralizing epitopes into recombinant vaccines will induce NAbs that produce additive, or optimally, synergistic effects.

When the immune response in rabbits was focused on the V3 epitope of the HIV gp120 envelope glycoprotein, NAbs were elicited with cross-clade neutralizing activity. Sera from animals primed with gp120 DNA and boosted with one or more V3-FPs carrying the consensus V3 sequences of clade A, B or C were able to neutralize 3 of 10 primary isolates, including those that are heterologous to the parental strains from which the immunogens were constructed, viruses from heterologous clades and viruses that contained the heterologous motif (GPGR (SEQ ID NO:17) or GPGQ (SEQ ID NO:18)) at the tip of the V3 loop.

Moreover, when tested against chimeric pseudoviruses carrying unmasked consensus V3 loops from several clades, GMT₉₀>1:100 were demonstrable against pseudoviruses carrying the consensus V3 sequences from clades A1, B, F, AE, and AG. Indeed, when one compares the results of immunization with gp120 DNA and V3-FPs to other DNA prime/Env boost regimens, the results with the former are at least as good, and often better, when tested against clade B primary isolates, and demonstrate greater breadth and potency against non-B viruses and pseudoviruses.

The current study represents a step forward in the pursuit of strong and broad NAb responses to HIV. Most studies to date in animals and humans either
(a) failed to elicit NAbs (Kothe, D L et al. (2006). Virology 352:438-49; Mulligan, M J et al. (2006). AIDS Res Hum Retroviruses 22:678-83),
(b) succeeded in inducing Abs that neutralize only T cell line-adapted viruses or viruses homologous to the strain or clade on which the immunogens were based (Gilbert, P B et al. (2005) J Infect Dis 191:666-77; Grundner, C et al. (2005). Virology 331:33-46; Rasmussen, R A et al. (2006) Vaccine 24:2324-32; Xu, R et al. (2006) Virology 349:276-89)
(c) elicited cross-clade NAbs with 50% neutralizing titers of only ˜1:5 (Mascola, J R et al., J Virol 79:771-9; Wu, L et al. (2006). Vaccine 24:4995-5002).

The use of DNA priming and protein boosts has proven to be one of the best regimens for inducing anti-Env Ab responses (Richmond et al., supra; Barnett et al., 1997, supra), and polyvalent vaccines based on the DNA prime/protein boost approach have proven to induce broader immune responses than similar monovalent vaccines (Chakrabarti et al., supra; Lian et al., supra). The present inventor have modified and extended previous work using the DNA prime/protein boost approach by using polyvalent combinations in both the prime and boost, and by focusing the Ab response on a single gp120 neutralizing epitope, the V3 loop. The results demonstrate that a cross-clade NAb response can be achieved using a clade C gp120 DNA prime and a boost in which the Ab response is focused on the V3 loops of clades A, B and C; this regimen resulted in a broad response in which heterologous primary isolates from two clades carrying the GPGR (SEQ ID NO:17) or GPGQ (SEQ ID NO:18) V3 motifs were neutralized, and pseudoviruses carrying the consensus V3 sequences from clades A, B, E, F and AG were also neutralized. As previously reported (Wang et al., 2005, supra; Wang et al., 2006, supra) very low levels of NAbs were induced by priming alone, but peak Ab responses were elicited only after the protein boosts (FIG. 3).

In the present studies, the most broadly NAbs were induced by priming with a clade C gp120 DNA and boosting with V3-FPs carrying the consensus sequences of clades A, B, and C (Group II-3: C_{Q}/ABC). This finding was supported by the higher levels in this group of both cross-clade binding and NAbs (Tables 4 and 5; FIGS. 5 and 6). This group is distinguished as the only one primed with the full dose of a gp120 DNA construct carrying the GPGQ V3 motif (SEQ ID NO:18). Interestingly, neither rabbits in Group II-2: A_{R}/ABC or Group II-4: A_{R}+C_{Q}/ABC produced Abs of comparable breadth or potency. A possible explanation is that, for these latter groups, priming was achieved using a construct carrying the GPGR V3 motif (SEQ ID NO:17) or using a priming dose that was “split” between DNA plasmids expressing the GPGR and the GPGQ V3 motifs. These results suggests the possibility that the GPGR immunogen is dominant over the GPGQ immunogen in eliciting the Ab responses when a combination of both are used for priming. Other explanations for these findings include the possibility that (a) priming with clade C gp120 DNA is superior to priming with clade A gp120 DNA, and/or (b) the observed differences were due to other factors contributed by the individual gp120 constructs used.

Since immunization regimens differ and methods for measuring neutralization vary, it is often difficult to compare results from various experiments conducted by different investigators. To facilitate comparison of the present results with previous experiments, a group of rabbits were included in Experiment 1 that were primed with gp120 DNA and boosted with gp120 protein—a control group immunized with a regimen similar to those reported by others used previously (Richmond et al. (1998), supra; Wang et al., 2005, supra). This group served as a standard for qualitative and quantitative comparisons. Based on comparison of the results from the sera of the control and experimental groups presented here, it appears that the immunofocusing vaccine regimens employed here are advantageous compared to results obtained with vaccines targeting the many epitopes of the HIV Env. For example, in rabbits primed with gp120_{JR-FL} or gp140_{JR-FL} DNA and boosted with Env_{JR-FL}, neutralizing Abs were induced to the relatively resistant homologous JR-FL strain and to SF162 but little or no neutralizing activity was detected against other clade B primary isolates or against primary isolates from other clades (Wang et al., 2005, supra). The results with the multiclade immunofocusing protocol of the present invention also exceed, in qualitative and quantitative terms, those recently published by Law et al., 2007, supra). In the latter report, rabbits were given four priming doses of codon-optimized JR-FL gp120 DNA and three boosts with a modified form of JR-FL gp120 in which the V1V2 loop was replaced with the gp41 MPER region containing two deleted residues immediately preceding the 4E10 epitope. Sera from these immunized rabbits displayed ND₅₀s against a psV carrying the Env of SF162 of 1:10-1:320, however 90% neutralization was never achieved. In contrast, the sera from animals primed and boosted according to this invention, displayed ND₅₀s against the SF162 psV that ranged from 1:190 to 1:3550, and ND₉₀s in all primed and boosted rabbits in the range of 1:14 to 1:190. The present results for the multiclade immunofocusing regimen also compare favorably, in terms of the titer and breadth of the response, with the results of previously published multiclade immunizations using immunogens that included all or most of the Env epitopes (Chakrabarti et al., supra; Lian et al., supra; Wang et al., 2006 supra).

The present invention represents a significant step forward by showing that, in focusing the immune response on a single neutralizing epitope, a functional Ab response is achieved that often better than (and at least comparable to) that induced by Env immunogens possessing a multitude of B cell epitopes. The present invention teaches that that focusing the immune response on a few, carefully selected neutralizing epitopes and optimizing the structure of these epitopes and the scaffolds on which they are presented, results in a stronger and broader neutralizing Ab response than that induced by Env proteins carrying the many epitopes of the Env.

Interestingly, the GPGR-based and multiclade immunization regimens used to prime and boost the immune response in the experiments described here resulted in approximately comparable neutralizing Ab responses against the Tier 1 clade B primary isolate BX08 and against the psV carrying the clade B V3 consensus sequence (Table II and FIGS. 5 and 6). In contrast, immunization with clade C gp120 DNA and the polyvalent combination of V3-FPs (Group II-3: C_{Q}/ABC) elicited the broadest and/or most potent response against the Tier 1 primary isolates DJ263 (CRF02_AG) which carries the GPGQ V3 motif (SEQ ID NO:18) and against the chimeric psVs carrying V3 loops with the GPGQ motif. These finding support prior results of the present inventor and colleagues that suggest an antigenic difference between viruses carrying the GPGR (SEQ ID NO:17) and GPGQ (SEQ ID NO:18) V3 motifs (Zolla-Pazner, S et al. (2004) AIDS Res Hum Retrovir 20:1254-80) and document that anti-V3 Ab responses induced by “GPGR viruses” favor neutralization of GPGR viruses but that anti-V3 Ab responses induced by non-B “GPGQ viruses” neutralize both GPGQ and GPGR viruses (Gorny et al., 2006, supra; Krachmarov et al., 2005, supra.)

Group II-3: C_{Q}/ABC is further distinguished as the only immunized group primed with the full dose of a gp120 DNA construct carrying the GPGQ V3 motif. Interestingly, neither rabbits in Group II-2: A_{R}/ABC or Group II-4: A_{R}+C_{Q}/ABC produced Abs of comparable breadth or potency. A possible explanation for this is that these two latter groups were primed, respectively, with A_{R}, a construct carrying the GPGR V3 motif or with A_{R}+C_{Q}, a priming dose containing half the dose of each prime relative to the dose of C_{Q}, the clade C (GPGQ) priming dose administered in Group II-3: C_{Q}/ABC. These results suggest that when a combination of GPGR and GPGQ immunogens are used for priming, the GPGR immunogen is dominant in eliciting Ab responses. Other explanations for these findings include the possibility that a clade C gp120 DNA prime is superior to a clade A gp120 DNA prime, and/or that the differences are due to other factors contributed by the individual gp120 constructs used.

The present results demonstrate that it is possible to focus the immune response on an epitope that elicits neutralizing Abs, in the present examples, the V3 loop. Thus, the majority of NAbs elicited by priming with a gp120 DNA and boosting with V3-FP were specific for V3. In contrast, only a minority of NAbs elicited by similar priming but boosting with gp120 protein were directed against V3 (FIG. 4 and Table 4). It is noteworthy that when a NAb response was elicited with the V3-FP boost, the cross-clade neutralizing activity could be significantly blocked by a single V3 peptide derived from the clade B consensus sequence. This stands in contrast to the work of Chakrabarti et al., supra in which immunization of guinea pigs was carried out with DNA encoding gp145ΔCFI of one or several clades and replication-defective recombinant adenoviruses encoding the gp140ΔCFI of the same strains. In these latter experiments, when the polyvalent regimen was used, weak cross-clade NAb responses were elicited (neutralizing titers never exceeded 1:5, and absorption with V3 peptides did not remove the neutralizing activity).

The present invention demonstrates the advantage of focusing the Ab response on a single protein domain or epitope that elicits NAbs, and the advantages of using polyvalent immunogens to increase the breadth of the Ab response. The results prove the principle that focusing the immune response on regions of the HIV-1 envelope that elicit NAbs are advantageous over the prior art practice of using Env immunogens that present a multitude of epitopes, the majority of which do not induce NAb responses.

This novel approach of using immunogenic/vaccine constructs that are capable of immunofocusing the anti-HIV-1 humoral immune response provides a platform for improving further both the strength and breadth of Ab responses to HIV-1 and to other pathogens as well. The present invention provides a basis and understanding for (a) defining the best combinations of parental viral strains from which to build the priming and boosting immunogens, (b) designing immunogens that will optimally present the neutralizing epitopes and produce Abs of higher titer and affinity, and (c) ultimately focusing the immune response on those epitopes which are known to induce protective Abs.

The references cited above are all incorporated by reference herein, whether specifically incorporated or not. Also incorporated by reference in its entirety is co-pending PCT Application PCT/US07/72660.

Having now fully described this invention, it will be appreciated by those skilled in the art that the same can be performed within a wide range of equivalent parameters, concentrations, and conditions without departing from the spirit and scope of the invention and without undue experimentation.

1. An immunogenic composition for boosting a broadly-neutralizing cross-clade anti-HIV antibody response in a subject who has been primed with an immunogen that primes for said antibody response, said composition comprising in unit dosage form one or more HIV-1 neutralizing epitopes each of which is in the form of a fusion protein that includes:
(a) a first fusion partner that comprises a neutralizing epitope of HIV-1 Env protein fused to
(b) a second fusion partner that is a polypeptide which, when fused to said first fusion partner, results in a fusion protein that adopts a conformation of said epitope that promotes an antibody response specific for said epitope upon immunization of a subject with said composition,
wherein administration to a primed subject of
(i) one unit dose of said immunogen, or
(ii) more than one unit dose of said immunogen simultaneously at different sites and/or sequentially,
results in a boosted broadly neutralizing cross-clade anti HIV-1 antibody response characterized by a serum neutralizing antibody titer that is increased at least 4-fold against at least two Tier 1 primary isolates from at least two different HIV-1 clades compared to the neutralizing titer of serum from similarly primed but non-boosted subjects.

2. The composition of claim 1 which, when administered to said primed subject, results in a serum neutralizing antibody titer of at least 1:20 against said Tier 1 primary isolates.

3. The composition of claim 1 wherein said unit dosage is between about 20 and 200 μg of said boosting immunogen.

4. The composition of claim 3 wherein the number of unit doses of the boosting immunogen given to result in said boosted neutralizing antibody titer results in a total administered dose of about 100 μg to about 200 μg of said boosting immunogen.

5. The composition of claim 1 wherein the first fusion partner comprises more than one neutralizing epitope of the Env protein.

6. The composition of claim 1 wherein, when the epitope is one that has a variable amino acid sequence among HIV-1 isolates in a clade, and the amino acid sequence of the first fusion partner is a consensus sequence of the epitope from a single clade of HIV-1 viruses.

7. The composition of claim 1, wherein
(A) the first fusion partner epitope has an amino acid sequence of a clade A, B or C virus, or
(B) the first fusion partner comprises more than one neutralizing epitope, each of which epitopes has an amino acid sequence of a clade A, B or C virus.

8. The composition of claim 7 wherein the amino acid sequence of the first fusion partner epitope or epitopes is a consensus sequence of the epitope from a clade A, B or C virus.

9. The composition of claim 1 wherein the neutralizing epitope is a V3 epitope and the fusion protein comprises said V3 epitope

10. The composition of claim 9 wherein the V3 epitope of the fusion protein comprises the amino acid sequence GPGR (SEQ ID NO:17) or GPGQ (SEQ ID NO:18.

11. (canceled)

12. The composition of claim 9 wherein the fusion protein includes two or more of the same or different V3 epitopes.

13. The composition of claim 9 that includes a mixture of two or more of:
(i) the fusion protein in which the first fusion partner has the V3 amino acid sequence of a clade A virus or the consensus V3 sequence of clade A viruses;
(ii) the fusion protein in which the first fusion partner has the V3 amino acid sequence of a clade B virus or the consensus V3 sequence of clade B viruses; or
(iii) the fusion protein in which the first fusion partner has the V3 amino acid sequence of a clade C virus or the consensus V3 sequence of clade C viruses.

14. (canceled)

15. The composition of claim 1 wherein the second fusion partner is MuLV gp70.

16. An immunogenic composition for both priming and boosting a broadly-neutralizing, cross-clade anti-HIV-1 antibody response specific for a selected HIV-1 neutralizing peptide epitope, the composition comprising:
(a) a specific priming immunogen for the peptide epitope in unit dosage form that comprises DNA encoding an HIV-1 polypeptide in which an amino acid sequence of the epitope is present; and
(b) in unit dosage form, a specific boosting immunogen specific for the epitope, which boosting immunogen is in the form of a fusion protein that includes:
(i) a first fusion partner that comprises a neutralizing epitope of HIV-1 peptide Env protein fused to
(ii) a second fusion partner that, when fused to said first fusion partner, results in a fusion protein that adopts a conformation of said epitope that promotes an antibody response specific for said epitope when the boosting immunogen is administered to a subject that has been primed with said priming immunogen.

17. The composition of claim 16, wherein
(1) priming of a subject with one or more unit doses of said priming immunogen, followed by
(2) boosting the subject with
(i) one unit dose of said boosting immunogen, or
(ii) more than one unit dose of said immunogen administered simultaneously at different sites and/or administered sequentially
results in a boosted broadly neutralizing cross-clade anti HIV-1 antibody response characterized by a serum neutralizing antibody titer that is increased at least 4-fold against at least two Tier 1 primary isolates from at least two different HIV-1 clades compared to the neutralizing titer of serum from either similarly primed but non-boosted subjects, or unprimed but similarly boosted subjects.

18. The composition of claim 16, wherein the unit dosage of the boosting immunogen is between about 20 and 200 μg of said fusion protein.

19. The composition of claim 17 wherein the number of unit doses of the boosting immunogen required to result in said boosted neutralizing antibody titer results in a total administered dose of about 100 μg to about 200 μg of said boosting immunogen.

20. The composition of claim 16, wherein the unit dosage of the priming immunogen is about 1 μg to about 100 μg of said DNA.

21. (canceled)

22. The composition of claim 16 wherein, when the epitope is one that has a variable amino acid sequence among HIV-1 isolates (i) in a clade and/or (ii) among clades, the amino acid sequence of the first fusion partner is a consensus sequence of the epitope from a single clade of the virus.

23. The composition of claim 16, wherein first fusion partner has an amino acid sequence of a clade A, B or C virus or a consensus sequence of the epitope from a clade A, B or C virus.

24. The composition of claim 16 wherein the neutralizing epitope is a V3 epitope and the fusion protein is a V3 fusion protein, wherein the boosting immunogen optionally comprises a combination of V3 fusion proteins or a V3 fusion protein that includes two or more of the same or different V3 epitopes.

25. The composition of claim 24 wherein the priming immunogen comprises
(A) env DNA encoding an Env protein bearing an amino acid sequence of GPGR (SEQ ID NO:17) corresponding to the tip of the V3 peptide loop, and/or
(B) env DNA encoding an Env protein bearing an amino acid sequence of GPGQ (SEQ ID NO:18) corresponding to the tip of the V3 peptide loop.

26. The composition of claim 24 wherein the V3 epitope of the fusion protein comprises the amino acid sequence GPGR (SEQ ID NO:17) or GPGQ (SEQ ID NO:18.

27. (canceled)

28. The composition of claim 24 wherein the fusion protein includes two or more of the same or different V3 epitopes

29. The composition of claim 24 wherein the V3 fusion protein combination of the boosting immunogen is a mixture of two or more of:
(i) a fusion protein in which the first fusion partner has the V3 amino acid sequence of a clade A virus or the consensus V3 sequence of clade A viruses;
(ii) a fusion protein in which the first fusion partner has the V3 amino acid sequence of a clade B virus or the consensus V3 sequence of clade B viruses;
(iii) a fusion protein in which the first fusion partner has the V3 amino acid sequence of a clade C virus or the consensus V3 sequence of clade C viruses.

30. (canceled)

31. The composition of claim 16 wherein the second fusion partner is MuLV gp70.

32. An immunogenic pharmaceutical composition comprising the immunogenic composition of claim 1 and an immunologically and pharmaceutically acceptable carrier or excipient.

33. A method of immunizing a mammalian subject to produce a broadly-neutralizing cross-clade anti-HIV antibody response specific for an HIV-1 neutralizing epitope, comprising administering, to a subject who has been primed with an immunogen that primes for said antibody response, one or more unit doses of an immunogenically-effective amount of said immunogenic booster composition of claim 1, wherein the immunization results in a boosted broadly neutralizing cross-clade anti HIV-1 antibody response in which a serum neutralizing antibody titer in said subject is increased at least 4-fold against at least two Tier 1 primary isolates each from at least two different HIV-1 clades compared to the neutralizing titer of serum from similarly primed but non-boosted subjects.

34. The method of claim 33 wherein said administration to said primed subject results in a serum neutralizing antibody titer of at least 1:20 against said Tier 1 primary isolates.

35. A method of immunizing a mammalian subject to produce a broadly-neutralizing cross-clade anti-HIV antibody response specific for an HIV-1 neutralizing epitope, comprising administering, to a subject an effective immunogenic amount of the composition of claim 16, which administering comprises:
(a) priming the subject with one or more unit doses of said priming immunogen; and
(b) between about one and about 12 weeks after said priming, boosting said subject with one or more simultaneous or sequential unit doses of an immunogenically effective amount of said boosting immunogen,
wherein the immunization results in a boosted, broadly neutralizing cross-clade anti HIV-1 antibody response in which a serum neutralizing antibody titer in said subject is increased at least 4-fold against at least two Tier 1 primary isolates each from at least two different HIV-1 clades compared to the neutralizing titer of serum from either similarly primed but non-boosted subjects, or unprimed but similarly boosted subjects.

36. The method of claim 35 wherein said priming and boosting results in a serum neutralizing antibody titer of at least 1:20 against said Tier 1 primary isolates.

37. The method of claim 33 further comprising administering an adjuvant or an immunostimulatory protein different from said fusion protein, before, during, or after said priming or said boosting.

38.-39. (canceled)

40. The method of claim 33 wherein the boosting immunogen is administered intradermally, subcutaneously or intramuscularly.

41. The method of claim 35, wherein the priming immunogen is administered by needle-less jet injection, intradermal injection, intramuscular injection, epidermal patch, epidermal abrasion, or gene gun delivery.

42. The method of claim 33 wherein the mammalian subject is a rodent, a rabbit, or a non-human primate.

43. The method of claim 33 wherein the mammalian subject is a human.

44. The method of claim 43, wherein the subject is susceptible to, or at risk of, HIV-1 infection.

45. The method of claim 43, wherein the subject is infected with HIV-1.

46. A kit comprising in separate compartments in close proximity therein:
(a) one or more unit dosages of the boosting immunogenic composition of claim 1, and
(b instructions for administering the boosting immunogenic composition to a subject for boosting said antibody response.

47. A kit comprising, in separate compartments in close proximity therein:
(a) one or more unit dosages of the priming immunogen of the composition of claim 16;
(b) one or more unit dosages of the boosting immunogen of the composition of claim 16; and
(c) instructions for administering the priming and the boosting immunogens and optionally, an adjuvant or immunostimulatory protein, to a subject for producing said antibody response.

48. (canceled)

49. An immunogenic pharmaceutical composition comprising the immunogenic composition of claim 16 and an immunologically and pharmaceutically acceptable carrier or excipient


SEQUENCE LISTING (18 sequences)

SEQ ID NO: 1 - protein, 35 aa, Artificial
  HIV-1 gp120 V3 region consensus sequence, clade B
CTRPNNNTRKSIHIGPGRAFYTTGEIIGDIRQAHC

SEQ ID NO: 2 - protein, 35 aa, Artificial
  HIV-1 gp120 V3 region consensus sequence, clade F
CTRPNNNTRKSIHIGPGQAFYATGEIIGDIRKAHC

SEQ ID NO: 3 - protein, 35 aa, Artificial
  HIV-1 gp120 V3 region consensus sequence, clade AE
CTRPSNNTRTSITIGPGQVFYRTGDIIGDIRKAYC

SEQ ID NO: 4 - protein, 35 aa, Artificial
  HIV-1 gp120 V3 region consensus sequence, clade A1
CTRPNNNTRKSIRIGPGQAFYATGDIIGDIRQAHC

SEQ ID NO: 5 - protein, 35 aa, Artificial
  HIV-1 gp120 V3 region consensus sequence, clade AG
CTRPNNNTRKSVRIGPGQTFYATGDIIGDIRQAHC

SEQ ID NO: 6 - protein, 35 aa, Artificial
  HIV-1 gp120 V3 region consensus sequence, clade C
CTRPNNNTRKSIRIGPGQTFYATGDIIGDIRQAHC

SEQ ID NO: 7 - protein, 35 aa, Artificial
  HIV-1 gp120 V3 region consensus sequence, clade H
CTRPNNNTRKSIHLGPGQAFYATGDIIGDIRQAHC

SEQ ID NO: 8 - DNA, 29 bp, Artificial
  synthetic
gtcgctccgctagcctgtgggtgaccgtg

SEQ ID NO: 9 - DNA, 36 bp, Artificial
  synthetic
acctacggatccttactgcaccactcttctcttggc

SEQ ID NO: 10 - DNA, 39 bp, Artificial
  synthetic
gtcgctccagctagcctgtgggtgaccgtgtactacggc

SEQ ID NO: 11 - DNA, 34 bp, Artificial
  synthetic
cgacggatccttactccaccacgcggcgcttggc

SEQ ID NO: 12 - protein, 23 aa, Artificial
  HIV-1 gp120 V3 region consensus sequence
TRPNNNTRKSIHIGPGRAFYTTG

SEQ ID NO: 13 - protein, 35 aa, Human immunodeficiency virus-1
  gp120 V3 region from clade A1
CTRPNNNTRKGIHIGPGRAIYATGDIIGDIRQAHC

SEQ ID NO: 14 - protein, 35 aa, Human immunodeficiency virus-1
  gp120 V3 region from clade C (strain 92BR025.9)
CTRPNNNTRKSIRIGPGQAFYATGEIIGDIRQAHC

SEQ ID NO: 15 - protein, 35 aa, Human immunodeficiency virus-1; identical to SEQ ID NO: 5
  gp120 V3 region from clade A (strain 92UG037.08)

SEQ ID NO: 16 - protein, 35 aa, Human immunodeficiency virus-1
  gp120 V3 region from clade B (strain JR-CSF)
CTRPSNNTRKSIHIGPGRAFYTTGEIIGDIRQAHC

SEQ ID NO: 17 - protein, 4 aa, Artificial
  synthetic
GPGR

SEQ ID NO: 18 - protein, 4 aa, Artificial
  synthetic
GPGQ